(19) United States
(12) Patent Application Publication
(10) Pub. No.: US 2010/0021472 A1
(43) Pub. Date: Jan. 28, 2010

(54) METHODS FOR DIAGNOSING AND TREATING CANCER

(76) Inventors: Geetha Srikrishna, San Diego, CA (US); Hudson H. Freeze, San Diego, CA (US); Suzanne Ostrand-Rosenberg, Baltimore, MD (US); Pratima Sinha, Baltimore, MD (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/510,053

(22) Filed: Jul. 27, 2009

Related U.S. Application Data
(60) Provisional application No. 61/083,841, filed on Jul. 25, 2008.

(51) Int. Cl. A61K 39/395

(52) U.S. Cl. 424/143.1; 424/172.1

(57) ABSTRACT

Aspects of the present invention relate to molecular biology and medicine. More specifically, some embodiments include methods for treating and/or diagnosing inflammation and/or cancer using agents that inhibit the binding of a pro-inflammatory protein or protein complex (e.g., S100A8 and/or S100A9) to a carboxylated glycan expressed on a myeloid (e.g., MDSC), monocytic, dendritic, endothelial, or tumor cell.

### CROSS-REFERENCE TO RELATED APPLICATIONS

This application claims the benefit of priority under 35 U.S.C. §119 to U.S. Provisional Patent Application No. 61/083,841 filed Jul. 25, 2008, and which is expressly incorporated herein by reference in its entirety.

### BACKGROUND OF THE INVENTION

1. Field of the Invention

Aspects of the present invention relate to molecular biology and medicine. More specifically, some embodiments include methods for treating or diagnosing inflammation or cancer using agents that inhibit the binding of a pro-inflammatory protein or protein complex to a carboxylated glycan expressed on a myeloid, monocytic, dendritic, endothelial, or tumor cell.

2. Description of the Related Art

Cancer is diagnosed in more than 1 million people every year in the United States alone. In spite of numerous advances in medical research, cancer remains the second leading cause of death in the United States, accounting for roughly 1 in every four deaths. Although numerous treatments are available for various cancers, many forms of cancer remain uncurable, untreatable, and/or become resistant to standard therapies.

Colorectal cancer (CRC) remains one of the most diagnosed and leading causes of cancer related deaths worldwide. Patients with inflammatory bowel disease (IBD) are at a higher risk for developing CRC than the general population. Several lines of evidence point to chronic inflammation of the colon as an important factor in the progression to CRC in IBD (reviewed in Itzkowitz et al. (2004) Am J Physiol Gastrointest Liver Physiol, 287, G7-17). However, the molecular basis of the association between inflammation and cancer remains poorly understood. Prolonged pro-inflammatory signaling and defective anti-inflammatory responses lead to a state of persistent inflammation. Inflammatory cells, particularly macrophages, produce soluble factors including cytokines, growth and angiogenic factors, and matrix metalloproteinases, creating a microenvironment that supports proliferation, invasion and metastasis of transformed cells (Mantovani, A. (2005) Nature, 435, 752-3; Condeelis et al. (2006) Cell, 124, 263-6). Chronic inflammation is a complex process that promotes carcinogenesis and tumor progression; however, the mechanisms by which specific inflammatory mediators contribute to tumor growth remain unclear.

Accordingly, many investigators seek to design and develop new molecular target-specific anti-inflammation and/or anticancer therapies so as to improve survival and the quality of life of patients suffering from these disorders.

### SUMMARY OF THE INVENTION

Embodiments disclosed herein concern methods of characterizing a protein receptor known as Receptor for Advanced Glycation End-products (RAGE) that has been modified with a carboxylated glycan. Some embodiments relate to a method of blocking the interaction of the modified RAGE with an S100 protein. The S100 protein can be, for example, S100A1, S100A2, S100A3, S100A4, S100A5, S100A6, S100A7, S100A8, S100A9, S100A10, S100A11, S100A12, S100A13, S100A14, S100A15, S100A16. The S100 protein can also be a heterodimer (e.g., S100A8/A9). The interaction between modified RAGE and S100 can be blocked, for example, by inhibitory agents such as antibodies (e.g., monoclonal or polyclonal antibodies) or small molecule inhibitors.

Some embodiments disclosed herein concern methods of treating inflammation and/or cancer (e.g., colon cancer) in a subject by administering to the subject an agent that inhibits binding of a pro-inflammatory protein or protein complex (e.g., an S100 protein such as S100A8 and/or S100A9) to a cell surface glycoprotein receptor. In some embodiments, the cell surface glycoprotein receptor can be RAGE. In some, embodiments, the cell surface glycoprotein receptor (e.g, RAGE) has been modified with a carboxylated glycan. In some embodiments, the pro-inflammatory protein can be an SiO0 protein such as S100A8 or S100A9. In some embodiments, the pro-inflammatory protein complex can be an S100A8/S100A9 heterodimer. In some embodiments, the inhibitory agent inhibits the binding of a pro-inflammatory protein or protein complex (e.g., S100A8 and/or S100A9) to a carboxylated glycan expressed on a myeloid (e.g., MDSC), monocytic, dendritic, endothelial, or tumor cell or on a receptor on the cell. Embodiments include methods of treating inflammation and/or cancer using inhibitory agents that inhibit the binding of S100A8/A9 to a subset of RAGE that contains a carboxylated glycan. Other embodiments include methods of treating inflammation and/or cancer using inhibitory agents that inhibit the binding of S100A8/A9 to MDSCs that express a carboxylated glycan. In some embodiments, the carboxylated glycan is expressed on a signaling receptor (e.g., RAGE). The inhibitory agent can be used to modulate the pro-inflammatory response. In some embodiments, the agent is a antibody (e.g., a monoclonal antibody or a polyclonal antibody). In some embodiments, the antibody is monoclonal antibody GB3.1. In some embodiments, the method further comprises providing a cancer therapy such as one or more chemotherapeutic agents, radiation, or one or more biological agents specific for a cancer cell. In some embodiments, the inhibitory agent can be used, for example, to modulate a tumor cell (e.g., by inhibiting proliferation of the tumor cell) or cause the death of a tumor cell (e.g. by apoptosis).

Some embodiments relate to methods of preventing cancer in a subject comprising identifying a subject at high risk for cancer (e.g., a subject suffering from colonic inflammation) and providing the subject an agent that binds to a cell surface glycoprotein receptor (e.g., RAGE) or a pro-inflammatory protein or protein complex (e.g., an S100 protein such as S100A8 and/or S100A9) wherein the binding of the agent to the cell surface glycoprotein receptor or pro-inflammatory protein inhibits the binding of the pro-inflammatory protein to the cell surface glycoprotein receptor. In some embodiments, the cell surface glycoprotein receptor can be RAGE. In some embodiments, the cell surface glycoprotein receptor (e.g, RAGE) has been modified with a carboxylated glycan. In some embodiments, the pro-inflammatory protein can be an S100 protein such as S100A8 or S100A9. In some embodiments, the pro-inflammatory protein complex can be an S100A8/S100A9 heterodimer. In some embodiments, the agent is an antibody (e.g., a monoclonal antibody or a polyclonal antibody). In some embodiments, the antibody is monoclonal antibody GB3.1. In some embodiments, the inhibitory agent inhibits the binding of a pro-inflammatory protein or protein complex (e.g., S100A8 and/or S100A9) to a carboxylated glycan expressed on a myeloid (e.g., MDSC), monocytic, dendritic, endothelial, or tumor cell or on a receptor on the cell. In some embodiments, the method further comprises providing a cancer therapy, such as one or more chemotherapeutic agents, radiation, or one or more biological agents specific for a cancer cell.

In some embodiments, the pro-inflammatory protein is a protein derived from the S100 family of proteins. This includes, for example, S100A8 and S100A9. The pro-inflammatory protein can also be a heterodimer (e.g., a heterodimer of S100A8 and S100A9 (S100A8/A9)).

The agent can be, for example, an antibody (e.g. a monoclonal or polyclonal antibody) or a small molecule inhibitor. The antibody can be mAbGB3.1. The antibody can be humanized. In addition, fragments and binding portions of such an antibody are contemplated.

Various embodiments disclosed herein relate to methods to diagnose cancers (e.g., human carcinoma tumors of epithelial origin) using an antibody to a carboxylated glycan. The antibody can be used, for example, to stain tissue from said human subject.

Other embodiments include treating a human subject with an inflammatory condition or cancer by administering an inhibitory agent that binds to myeloid-derived suppressor cells (MDSC) and prevents the accumulation of MDSC at the site of inflammation or tumors. The inhibitory agent can be for example, an antibody (e.g. a monoclonal or polyclonal antibody) or a small molecule inhibitor that binds to a carboxylated glycan on the cell surface of MDSC. The antibody can be humanized or fully human.

### BRIEF DESCRIPTION OF THE DRAWINGS

FIG. 1. A. Colon tumor cells express RAGE and mAbGB3.1 glycans. Cell membrane proteins from CT-26 cells (lanes 1, 4 and 7), HT-29 cells (lanes 2, 5 and 8) and Caco-2 cells (lanes 3,6 and 9) were examined for RAGE expression by western blot using anti-RAGE before deglycosylation (lanes 1 to 3, 20 μg protein/lane) and after deglycosylation (lanes 4 to 6, 20 μg protein/lane) and after mAbGB3.1 immunoprecipitation (lanes 7 to 9, immunoprecipitated from 1 mg of membrane proteins). B. Purification of bovine lung RAGE and mAbGB3.1 enrichment. Left panel: Protein stain by Coomassie Brilliant Blue of a representative RAGE preparation from bovine lung shows >98% purity. Middle panel: Western blot using anti-RAGE shows that purified RAGE carries Endo-H sensitive and PNGase F sensitive N-glycan chains. Right panel: Western blot using anti-RAGE shows that mAbGB3.1 immunoprecipitates a minor sub-population of RAGE C. Purified mouse S100A8/A9. S100A8/A9 complex was purified as described before, and 5 ug protein analyzed on 17% gels and purity confirmed by Coomassie Brilliant Blue. Arrow marks the position of covalently linked 26kDa dimer of S100A8/A9 D. S100A8/A9 complex binds purified RAGE. To determine saturation kinetics of binding of mouse S100A8/A9 to purified RAGE, increasing amounts of S100A8/A9 were added to total RAGE, mAbGB3.1 enriched RAGE, or RAGE deglycosylated using PNGase F under non-denaturing conditions that removed both N-glycans. RAGE on plate was quantified using anti-RAGE. Bound S100A8/A9 was quantified using anti S100A8 against standard S100A8/A9. Data were fitted to non-linear regression analysis using GRAPHPAD PRISM®. Each point is the mean±SD of two determinations.

FIG. 2. A. Binding of 1251 S100A8/A9 to CT-26 cells. Cells were incubated with increasing concentrations of 1251 S100A8/A9 for 1 h at 4° C. followed by washing and cell lysis, and cell bound radioactivity was measured using a gamma counter. Saturation binding kinetic analysis was performed using GRAPHPAD PRISM®. Values represent mean±SD of two determinations B. Inhibition of binding of 125I S100A8/A9 to CT-26 cells. Cells were incubated with 125I S100A8/A9 (20 nM) in the presence or absence of mAbGB3.1, anti-RAGE or anti-S100A8 (10 fold molar excess) or cold ligand (50 fold molar excess). Cell bound radioactivity was determined as above. Data represent mean±SD of two determinations (* P≦0.05 and ** P≦0.01). C. S100A8/A9 induces NF-κB dependent transcription. CT-26 cells were transiently transfected with plasmids containing firefly luciferase reporter gene under a promoter containing NF-κB binding site and Renilla luciferase construct as an internal control. Transfected cells were stimulated with S100A8/A9 in presence or absence of inhibitors. Cell lysates were assayed for luciferase activity. Values are represented as ratio of firefly luciferase activity over Renilla luciferase (fold induction relative to unstimulated cells). Each value is the mean±S.D of two determinations (* P≦0.05 and ** P≦0.01). D. S100A8/A9 proteins stimulate colon cancer cell proliferation. CT-26 cells were incubated with increasing concentrations of S100A8/A9 in the presence or absence of mAbGB3.1, control antibody or anti-RAGE. At low concentrations S100A8/A9 stimulated cell proliferation that was blocked by mAbGB3.1 and anti-RAGE. S100A8/A9 induced growth was not dependent on time or concentration as seen earlier with other tumor cells.

FIG. 3. Schematic representation of the experimental protocol used to induce CAC. Mice were injected intraperitoneally with a single dose of AOM followed a week later by 2% DSS in drinking water for five days and were sacrificed at 2, 6, 12 weeks after initiation of disease. For each time point, four groups of mice (n=4 each group) were either untreated or received mAbGB3.1 or a non-specific control antibody weekly in preventive and therapeutic protocols as indicated. For each time point one group of four mice that did not receive AOM-DSS served as controls. Separate groups of wild type and RAGE−/— mice (n=4 per group) were subjected to the same protocol and sacrificed at 2 wks, 6 wks or 20 wks after DSS.

FIG. 4. A. mAbGB3.1 administration reduces colonic inflammation (6 wks) and incidence of tumors (12 wks in preventive and therapeutic protocols) in mice treated with AOM-DSS. Mice were administered with mAbGB3.1 or a control antibody as described in FIG. 4. Colonic inflammation, dysplasia and adenomas were evaluated using established criteria. (n=4 per group per time point, ** P≦0.01). B. TNFα and IL-6 were measured in sera of mice at different time points (n=4 per group per time point, ** P≦0.01). C. Colonic inflammation in RAGE^{+/+} and RAGE^{-/-} mice 2 weeks after AOM-DSS evaluated using established criteria. D. Colonic tumor incidence in RAGE^{+/+} and RAGE^{-/-} mice 20 weeks after AOM-DSS.

FIG. 5. mAbGB3.1 reactivity of purified RAGE was determined by ELISA: mAbGB3.1 enriched RAGE shows 10 fold increase in mAbGB3.1 reactivity compared to total unfractionated RAGE. Most of the binding is lost upon deglycosylation by PNGaseF. mAbGB3.1 reactivity is defined in arbitrary units (1 unit=mAbGB3.1 reactivity of 1 ng RAGE under standard ELISA conditions). Each point is the mean±SD of three determinations.

FIG. 6. Colonic inflammation is reduced in RAGE^{-/-} 6 wks after initiation of disease. Colons isolated from the mice were fixed, H&E stained and inflammation were evaluated using established criteria. (n=4 per group per time point, * P=0.05).

FIG. 7. MDSC present in the bone marrow, blood, spleen, or lungs of BALB/c mice with the 4T1 mammary carcinoma are Gr1^{hi}CD11b^{hi}F4/80⁻IL-4Ra^{+/-} CD80⁺Arginase^{hi} and have a mixed nuclear phenotype. A. Spleens, blood, bone marrow, and metastatic lung from BALB/c mice with 33 day primary tumors (11.5 mm in diameter) were harvested and the cells were stained with antibodies to Gr1, CD11b, F4/80, IL-4Rα, CD80, arginase, iNOs, Ly-6C, and Ly-6G. Gr1⁺CD11b⁺ populations were gated and further analyzed by flow cytometry for F4/80, IL-4Rα, CD80, arginase, or iNOS. B. CD11b⁺ cells of panel A were gated and analyzed for Ly6G and Ly6C. C. Cell populations from A were stained with Diff-Quik and analyzed by microscopy. Each panel shows representative cells assembled from 3-6 fields per MDSC sample. D. Purified Gr1⁺CD11b⁺ cells from the spleen, bone marrow, blood, or metastatic lungs were co-cultured with transgenic CD4⁺D011.10 or CD8⁺Clone 4 splenocytes plus OVA₃₂₃₋₃₃₉ or HA₅₁₈₋₅₂₆ peptides, respectively, and T cell activation measured by tritiated thymidine uptake.

FIG. 8. MDSC have glycoprotein receptors for and bind S100A8/A9 proteins. A. Leukocytes from the blood of tumor-free or 4T1-tumor-bearing BALB/c mice were stained with Gr1, CD11b, and GB3.1 mAbs. Gr1⁺CD11b⁺ cells were gated and analyzed for expression of GB3.1 receptors. B. Cell membranes of MDSC derived from 4T1 tumor bearing mice were incubated with increasing concentrations of ¹²⁵I-S100A8/A9 proteins for 1 h at 4° C. followed by washing, and membrane bound radioactivity was measured. Saturation binding kinetic analysis was performed using GRAPHPAD PRISMO. Values represent mean±SD of two independent experiments. C. Inhibition of binding of ¹²⁵I-S100A8/A9 to MDSC. MDSC membranes were incubated with ¹²⁵I-S100A8/A9 proteins (20 nM) in the presence or absence of mAbGB3.1, anti-RAGE, anti-S100A8 (10 fold molar excess), cold ligand (100 fold molar excess), or isotype control antibodies. Membrane-bound radioactivity was determined as in panel A. Data are from one of two independent experiments.

FIG. 9. MSDC express and secrete S100A8/A9 proteins. A. Circulating white blood cells pooled from five 4T1 tumor-bearing mice (left panel) or pooled white blood cells purified by MACS-sorting for Gr-1 from 60 tumor-free mice (right panel) were stained with fluorescent antibodies to Gr1 and CD11b and analyzed by flow cytometry. B. MDSC contain elevated levels of mRNA for pro-inflammatory mediators. Real-time RT-PCR was performed on mRNA obtained from the purified cells of panel A. Expression levels are presented as the number of threshold cycles±SD needed to detect a product. Fold increase is the relative increase in MDSC from tumor-bearing mice vs. tumor-free mice. Values for the house-keeping genes (HG) are the average of five genes: GAPDH, Hprt1, Hsp90ab1, Actb, and Gusb. CXCL1 is included as an example of a poorly expressed gene. C. Lysates of the Gr1⁺CD11b⁺ cells shown in panel A were electrophoresced on 12% SDS-PAGE gels, and western blotted with antibodies to S100A8, S100A9, HMGB1, and RAGE. D. MDSC purified from BALB/c 4T1 tumor-bearing or from tumor-free mice were cultured for 16 hr. and the supernatants were assayed by ELISA for S100A8/A9 proteins. Data are from one of two independent experiments. E. NF-κB is phosphorylated in MDSC following binding of S100A8/A9 proteins. MDSC from tumor-bearing or tumor-free mice were co-cultured with S100A8/A9 or TNFα for 10 minutes and cell lysates were screened for phosphorylated-NF-κB p65 (ser536) by ELISA. Data are from one of two independent experiments.

FIG. 10. mAbGB3.1 reduces MDSC and S100A8/A9 proteins in the blood of tumor-bearing mice. A. mAbGB3.1 treatment reduces serum levels of MDSC in tumor-bearing mice. BALB/c mice were inoculated on day 0 with 4T1 tumor cells, their primary tumors were removed on day 20, and GB3.1 or control antibody treatment was started 3 days later (day 24) and continued once weekly. Mice were bled 72 hr after each antibody treatment, and their white blood cells stained for Gr1 and CD11b. At the time of surgery primary tumor diameters were 5.09±0.76 and 5.36±0.68 mm, and percent Gr1⁺CD11b⁺ MDSC in the blood were 41.25±3.98 and 44.65±6.60 percent, for the mAbGB3.1 and control antibody-treated groups, respectively. Data are the average±SD of four mice in each group. Experiment was terminated on day 42 when mice were moribund. Data are from one of two independent experiments. B. mAbGB3.1 treatment reduces serum levels of S100A8/A9 proteins. GB3.1 and control antibody-treated post-surgery mice from panel A were bled on day 41 (20 days after surgery), and the serum assayed by ELISA for S100A8/A9 proteins. Serum from tumor-free (naive) mice was included for comparison. C. Serum S100A8/A9 levels are proportional to the amount of circulating MDSC. Tumor-bearing mice were bled and serum levels of S100A8/A9 were determined by ELISA and the percent of MDSC determined by flow cytometry. Data are from one of two independent experiments.

FIG. 11. 4T1 tumor cells secrete S100A8/A9 proteins which are chemotactic for MDSC. A. Conditioned media from 4T1 tumor cells was tested by ELISA for S100A8/A9. B. Blood MDSC (>90% Gr1⁺CD11b⁺ cells) were tested by chemotaxis assay for their migration in response to 4T1 CM. Data is pooled from two independent experiments.

FIG. 12. Blocking S100A8/A9 binding reduces MDSC accumulation in vivo in tumor-bearing mice but does not alter immunosuppressive activity of MDSC on a per cell basis. A. Average number of Gr1⁺CD11b⁺ cells counted in three high power fields of lymph nodes and spleen for untreated, mAb GB3.1-treated, and control antibody-treated groups. B. CD4⁺ DO11.10 or CD8⁺ Clone 4 transgenic splenocytes were co-cultured with OVA or HA peptide, respectively, in the presence or absence of graded doses of MACS sorted splenic Gr1⁺CD11b⁺ MDSC (>90% Gr1⁺CD11b⁺) from mAbGB3.1 or control mAb-treated post-surgery BALB/c mice.

### DETAILED DESCRIPTION OF CERTAIN EMBODIMENTS

As discussed above, some patients with inflammatory bowel diseases (IBD) are at increased risk for colorectal cancer, but the molecular mechanisms linking inflammation and cancer are not well defined.

Embodiments described herein relate to the unexpected discovery that carboxylated glycans are expressed on a subpopulation of receptor for advanced glycation end-products (RAGE) on colon cancer cells and mediate S100A8/A9 binding to RAGE. Colon tumor cells express binding sites for S100A8/A9 and binding leads to activation of NF-κB and tumor cell proliferation. Also disclosed herein is the finding that binding, downstream signaling and tumor cell proliferation were blocked by mAbGB3.1, an anti-carboxylate glycan antibody, and by anti-RAGE. In human colon tumor tissues and in a mouse model of CAC, myeloid progenitors expressing S100A8 and S100A9 infiltrated regions of dysplasia and adenoma. mAbGB3.1 administration markedly reduced chronic inflammation and tumorigenesis in the mouse model of CAC and RAGE-deficient mice were resistant to the onset of CAC. These findings show that RAGE, carboxylated glycans and S100A8/A9 play essential roles in tumor-stromal interactions leading to inflammation associated colon carcinogenesis.

Additional embodiments relate to the unexpected discovery that myeloid derived suppressor cells (MDSC) from tumor-bearing mice not only have receptors for S100A8/A9, but also synthesize and secrete these proteins, providing an autocrine pathway for MDSC accumulation. Treatment of tumor-bearing mice with the mAbGB3.1, which binds to carboxylated N-glycans on cell surface receptors, blocked S100A8/A9 binding and signaling, reduced serum levels of S100A8/A9, and reduced the accumulation of MDSC in the blood and secondary lymphoid organs. Therefore, S100A8/A9 proteins are another class of pro-inflammatory mediators that complicate cancer immunotherapy strategies by promoting the accumulation of MDSC that block tumor immunity. Accordingly, drugs and other agents that target this pathway may reduce MDSC levels and be useful therapeutic agents and may be useful in conjunction with active immunotherapy in cancer patients.

As described herein, it is intended that where a range of values is provided, it is understood that each intervening value, to the tenth of the unit of the lower limit unless the context clearly dictates otherwise, between the upper and lower limit of that range and any other stated or intervening value in that stated range is encompassed within the embodiments. The upper and lower limits of these smaller ranges may independently be included in the smaller ranges is also encompassed within the embodiments, subject to any specifically excluded limit in the stated range. Where the stated range includes one or both of the limits, ranges excluding either both of those included limits are also included in the embodiments.

Unless defined otherwise, all technical and scientific terms used herein have the same meaning as commonly understood by one of ordinary skill in the art to which the embodiments belong. Although any methods and materials similar or equivalent to those described herein may also be used in the practice or testing of the embodiments, the preferred methods and materials are now described. All publications mentioned herein are expressly incorporated by reference in their entireties.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

In some contexts, the terms “individual,” “host,” “subject,” and “patient” are used interchangeably to refer to an animal that is the object of treatment, observation and/or experiment. “Animal” includes vertebrates and invertebrates, such as fish, shellfish, reptiles, birds, and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

In some contexts, the terms “ameliorating,” “treating,” “treatment,” “therapeutic,” or “therapy” do not necessarily mean total cure or abolition of the disease or condition. Any alleviation of any undesired signs or symptoms of a disease or condition, to any extent, can be considered amelioration, and in some respects a treatment and/or therapy.

The term “therapeutically effective amount/dose” or “inhibitory amount” is used to indicate an amount of an active compound, or pharmaceutical agent, that elicits a biological or medicinal response. This response may occur in a tissue, system, animal or human and includes alleviation of the symptoms of the disease being treated. For example, with respect to the treatment of cancer, a therapeutically effective amount preferably refers to the amount of a therapeutic agent that decreases the rate of tumor growth, decreases tumor mass, decreases the number of metastases, increases time to tumor progression, or increases survival time by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The term “nucleic acids”, as used herein, may be DNA or RNA or modified versions thereof. Nucleic acids may also include modified nucleotides that permit correct read through by a polymerase and do not alter expression of a polypeptide encoded by that nucleic acid. The terms “nucleic acid” and “oligonucleotide” are used interchangeably to refer to a molecule comprising multiple nucleotides. As used herein, the terms refer to oligoribonucleotides as well as oligodeoxyribonucleotides. The terms shall also include polynucleosides (e.g., a polynucleotide minus the phosphate) and any other organic base containing polymer. Nucleic acids include vectors, e.g., plasmids, as well as oligonucleotides. Nucleic acid molecules can be obtained from existing nucleic acid sources, but are preferably synthetic (e.g., produced by oligonucleotide synthesis).

The phrase “nucleotide sequence” includes both the sense and antisense strands as either individual single strands or in the duplex.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It being further understood that the sequence includes the degenerate codons of the native sequence or sequences which may be introduced to provide codon preference in a specific host cell.

By “DNA” is meant a polymeric form of deoxyribonucleotides (adenine, guanine, thymine, or cytosine) in double-stranded or single-stranded form, either relaxed or supercoiled. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (e.g., the strand having the sequence homologous to the mRNA). The term captures molecules that include the four bases adenine, guanine, thymine, or cytosine, as well as molecules that include base analogues, which are known in the art.

A “gene” or “coding sequence” or a sequence, which “encodes” a particular protein, is a nucleic acid molecule which is transcribed (in the case of DNA) and translated (in the case of mRNA) into a polypeptide in vitro or in vivo when placed under the control of appropriate regulatory or control sequences. The boundaries of the gene are determined by a start codon at the 5′ (amino) terminus and a translation stop codon at the 3′ (carboxy) terminus. A gene can include, but is not limited to, CDNA from prokaryotic or eukaryotic mRNA, genomic DNA sequences from prokaryotic or eukaryotic DNA, and even synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present so long as the selected coding sequence is capable of being replicated, transcribed and translated in an appropriate host cell.

The term “promoter region” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence.

The term “operably linked” refers to an arrangement of elements, wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

For the purpose of describing the relative position of nucleotide sequences in a particular nucleic acid molecule throughout the instant application, such as when a particular nucleotide sequence is described as being situated “upstream,” “downstream,” “5′,” or “3′” relative to another sequence, it is to be understood that it is the position of the sequences in the non-transcribed strand of a DNA molecule that is being referred to as is conventional in the art.

The terms “polypeptide,” “peptide,” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins. Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (e.g., 10 kDa) and/or when it cannot be produced by recombinant techniques (e.g., not encoded by a nucleic acid sequence) and therefore involves different chemistry. Solid phase polypeptide synthesis procedures are well known in the art and further described by John Morrow Stewart and Janis Dillaha Young, Solid Phase Peptide Syntheses (2nd Ed., Pierce Chemical Company, 1984). Synthetic polypeptides can optionally be purified by preparative high performance liquid chromatography [Creighton T. (1983) Proteins, structures and molecular principles. WH Freeman and Co. N.Y.], after which their composition can be confirmed via amino acid sequencing. In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The term “homology” refers to the percent of identity between two polynucleotide or two polypeptide moieties. The correspondence between the sequence from one moiety to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions, which form stable duplexes between homologous regions, followed by digestion with single-stranded-specific nuclease(s), and size determination of the digested fragments. Two DNA, or two polypeptide sequences are “substantially homologous” to each other when at least about 80%, preferably at least about 90%, and most preferably at least about 95% of the nucleotides or amino acids match over a defined length of the molecules, as determined using the methods above.

By “isolated” when referring to a nucleotide sequence, is meant that the indicated molecule is present in the substantial absence of other biological macromolecules of the same type. Thus, an “isolated nucleic acid molecule, which encodes a particular polypeptide,” refers to a nucleic acid molecule, which is substantially free of other nucleic acid molecules that do not encode the subject polypeptide; however, the molecule may include some additional bases or moieties, which do not deleteriously affect the basic characteristics of the composition.

The terms “vector”, “cloning vector”, “expression vector”, and “helper vector” mean the vehicle by which a DNA or RNA sequence (e.g., a foreign gene) can be introduced into a host cell, so as to promote expression (e.g., transcription and/or translation) of the introduced sequence. Vectors include plasmids, phages, viruses, pseudoviruses, etc. The phrase “gene transfer” or “gene delivery” refers to methods or systems for reliably inserting foreign DNA into host cells.

As used herein, the term “transfection” is understood to include any means, such as, but not limited to, adsorption, microinjection, electroporation, lipofection and the like for introducing an exogenous nucleic acid molecule into a host cell. The term “transfected” or “transformed”, when used to describe a cell, means a cell containing an exogenously introduced nucleic acid molecule and/or a cell whose genetic composition has been altered by the introduction of an exogenous nucleic acid molecule.

The term “hyperproliferative disease,” as used herein, refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The term “neoplastic disease,” as used herein, refers to any abnormal growth of cells being either benign (non-cancerous) or malignant (cancerous).

The term “anti-neoplastic agent,” as used herein, refers to any compound that retards the proliferation, growth, or spread of a targeted (e.g., malignant) neoplasm.

The term “apoptosis modulating agents,” as used herein, refers to agents which are involved in modulating (e.g., inhibiting, decreasing, increasing, promoting) apoptosis. Examples of apoptosis modulating agents include proteins and nucleic acids, which comprise a death domain or encode a death domain such as, but not limited to, Fas/CD95, TRAMP, TNF RI, DR1, DR2, DR3, DR4, DR5, DR6, FADD, and RIP. Small RNAs such as MIR RNAs can also be apoptosis modulating agents (e.g., MIR-34a). Other examples of apoptotic modulating agents include, but are not limited to, TNF-alpha, Fas ligand, antibodies to Fas/CD95 and other TNF family receptors, TRAIL, antibodies to TRAILR1 or TRAILR2, Bcl-2, p53, BAX, BAD, Akt, CAD, PI3 kinase, PP1, and caspase proteins. Modulating agents broadly include agonists and antagonists of TNF family receptors and TNF family ligands. Apoptosis modulating agents may be soluble or membrane bound (e.g. ligand or receptor). Preferred apoptosis modulating agents are inducers of apoptosis, such as TNF or a TNF-related ligand, particularly a TRAMP ligand, a Fas/CD95 ligand, a TNFR-1 ligand, or TRAIL.

The terms “anticancer agent” and “anticancer drug,” as used herein, refer to any therapeutic agents (e.g., chemotherapeutic compounds and/or molecular therapeutic compounds), radiation therapies, or surgical interventions, used in the treatment of hyperproliferative diseases such as cancer (e.g., in mammals).

The term “inflammation” as used herein refers to all categories of inflammation, including localized manifestations and systemic inflammation; inflammation that is categorized temporally, e.g., chronic inflammation and acute inflammation; inflammation that is categorized in terms of its severity, e.g., mild, moderate, or severe; and inflammation that is a symptom or a result of a disease state or syndrome. Inflammation, as used herein, can be characterized at the “whole body” level as several localized manifestations, including hemodynamic disorders (e.g., hyperemia and edema), pain, temperature increment, and functional lesion. All manifestations may be observed in certain instances, although any particular manifestation may not always be present in all instances. Concomitant cellular and molecular level changes that characterize inflammation may include leukocyte extravasation and platelet aggregation. Molecular level changes which characterize inflammation may include activation of at least three plasma defense systems and synthesis of cytokines and eicosanoids.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, “carrier” includes any and all solvents, dispersion media, vehicles, coatings, diluents, antibacterial and antifumgal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

As used herein, the term “heterologous sequence or gene” means a nucleic acid (RNA or DNA) sequence, which is not naturally found in association with the nucleic acid sequences of the specified molecule.

The section below provides greater detail on some approaches that can be used to prepare inhibitory agents that inhibit the binding of a pro-inflammatory protein or protein complex (e.g., S100A8 and/or S100A9) to a carboxylated glycan expressed on a myeloid (e.g., MDSC), monocytic, dendritic, endothelial, or tumor cell. For example, embodiments include approaches that can be used to prepare agents that inhibit the binding of S100A8/A9 to a subset of RAGE that contains a carboxylated glycan. In addition, embodiments include approaches that can be used to prepare agents that inhibit the binding of S100A8/A9 to MDSCs that express a carboxylated glycan. These agents may be useful for the treatment of inflammation and/or cancer (e.g., colon cancer).

### Inhibitory Agents

Embodiments disclosed herein concern methods of treating inflammation, methods of reducing the proliferation of cancer cell, methods of ameliorating cancer or a disorder associated with cancer, methods of killing cancer cells, and methods of treating a patient suffering from cancer and/or inflammation, by providing to a patient identified as one in need of a reduction in the proliferation of cancer cells and/or inflammation, an amelioration of inflammation, cancer and/or a disorder associated with cancer, a killing of cancer cells or a cancer treatment a composition that comprises an amount of an inhibitory agent sufficient to treat inflammation, reduce the proliferation of cancer cells, ameliorate inflammation, cancer and/or a disorder associated with cancer, kill cancer cells or treat the cancer. The identification of patents for such treatments can be accomplished by diagnostic or clinical approaches as known in the art. The inhibitory agent can be an antibody, a polypeptide (e.g., a dominant negative peptide). Additionally, the inhibitory agent may be a chemical inhibitor such as a small molecule, e.g., chemical molecules with a low molecular weight (e.g; a molecular weight below 2000 daltons).

### Preparation of Peptides and Polypeptides

In some embodiments, the inhibitory agent can be a polypeptide (e.g., a dominant negative peptide or an antibody). Polypeptides can be produced via several methods known in the art (e.g., synthetically or via recombinant methods).

In some embodiments, the method of making the polypeptides or fragments thereof is to clone a polynucleotide comprising the cDNA of the gene into an expression vector and culture the cell harboring the vector so as to express the encoded polypeptide, and then purify the resulting polypeptide, all performed using methods known in the art as described in, for example, Marshak et al., “Strategies for Protein Purification and Characterization. A laboratory course manual.” CSHL Press (1996). (in addition, see Bibl Haematol. 1965; 23:1165-74 Appl Microbiol. 1967 July; 15(4):851-6; Can J. Biochem. 1968 May; 46(5):441-4; Biochemistry. 1968 July; 7(7):2574-80; Arch Biochem Biophys. 1968 Sep. 10; 126(3):746-72; Biochem Biophys Res Commun. 1970 Feb. 20; 38(4):825-30).).

The expression vector can include a promoter for controlling transcription of the heterologous material and can be either a constitutive or inducible promoter to allow selective transcription. Enhancers that can be required to obtain necessary transcription levels can optionally be included. The expression vehicle can also include a selection gene.

Vectors can be introduced into cells or tissues by any one of a variety of methods known within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al. (1986).

### Preparation of Antibodies

In some embodiments, the inhibitory agent can be an antibody. Antibodies may be prepared using an intact polypeptide or fragments containing smaller polypeptides as the immunizing antigen. For example, it may be desirable to produce antibodies that specifically bind to the N- or C-terminal or any other suitable domains of a protein. The polypeptide used to immunize an animal can be derived from translated cDNA or chemical synthesis and can be conjugated to a carrier protein, if desired. Such commonly used carriers which are chemically coupled to the polypeptide include keyhole limpet hemocyanin (KLH), thyroglobulin, bovine serum albumin (BSA) and tetanus toxoid. The coupled polypeptide is then used to immunize the animal.

If desired, polyclonal or monoclonal antibodies can be further purified, for example by binding to and elution from a matrix to which the polypeptide or a peptide to which the antibodies were raised is bound. Those skilled in the art know various techniques common in immunology for purification and/or concentration of polyclonal as well as monoclonal antibodies (Coligan et al, Unit 9, Current Protocols in Immunology, Wiley Interscience, 1994).

Methods for making antibodies of all types, including fragments, are known in the art (See for example, Harlow and Lane, Antibodies: A Laboratory Manual, Cold Spring Harbor Laboratory, New York (1988)). Methods of immunization, including all necessary steps of preparing the immunogen in a suitable adjuvant, determining antibody binding, isolation of antibodies, methods for obtaining monoclonal antibodies, and humanization of monoclonal antibodies are all known to the skilled artisan

The antibodies may be humanized antibodies or human antibodies. Antibodies can be humanized using a variety of techniques known in the art including CDR-grafting (EP239,400: PCT publication WO0.91/09967; U.S. Pat. Nos. 5,225,539; 5,530,101; and 5,585,089, veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,332).

The monoclonal antibodies as defined include antibodies derived from one species (such as murine, rabbit, goat, rat, human, etc.) as well as antibodies derived from two (or more) species, such as chimeric and humanized antibodies.

Completely human antibodies are particularly desirable for therapeutic treatment of human patients. Human antibodies can be made by a variety of methods known in the art including phage display methods using antibody libraries derived from human immunoglobulin sequences. See also U.S. Pat. Nos. 4,444,887 and 4,716,111; and PCT publications WO 98/46645, WO 98/50433, WO 98/24893, WO 98/16654, WO 96/34096, WO 96/33735, and WO 91/10741, each of which is incorporated herein by reference in its entirety.

Additional information regarding all types of antibodies, including humanized antibodies, human antibodies and antibody fragments can be found in WO 01/05998, which is incorporated herein by reference in its entirety.

The polypeptides employed in embodiments disclosed herein may also be modified, optionally chemically modified, in order to improve their therapeutic activity. “Chemically modified”—when referring to the polypeptides, refers to a polypeptide where at least one of its amino acid residues is modified either by natural processes, such as processing or other post-translational modifications, or by chemical modification techniques which are well known in the art. Among the numerous known modifications typical, but not exclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

Additional possible polypeptide modifications (such as those resulting from nucleic acid sequence alteration) include substitutions, deletions, and insertions.

A “conservative substitution” refers to the substitution of an amino acid in one class by an amino acid of the same class, where a class is defined by common physicochemical amino acid side chain properties and high substitution frequencies in homologous polypeptides found in nature, as determined, for example, by a standard Dayhoff frequency exchange matrix or BLOSUM matrix.

A “non-conservative substitution” refers to the substitution of an amino acid in one class with an amino acid from another class; for example, substitution of an Ala, a class II residue, with a class III residue such as Asp, Asn, Glu, or Gln.

A “deletion” refers to a change in either nucleotide or amino acid sequence in which one or more nucleotides or amino acid residues, respectively, are absent.

An “insertion” or “addition” refers to a change in a nucleotide or amino acid sequence which has resulted in the addition of one or more nucleotides or amino acid residues, respectively, as compared to the naturally occurring sequence.

A “substitution” refers to the replacement of one or more nucleotides or amino acids by different nucleotides or amino acids, respectively. As regards amino acid sequences the substitution may be conservative or non-conservative.

Embodiments disclosed herein also include polypeptides (e.g., dominant negative polypeptides or antibodies) that can have the following degrees of homology or identity to an inhibitory polypeptide: 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. Candidate polypeptides having greater than or equal to 35% homology or identity to an inhibitory polypeptide can be identified by methods known in the art and can be subsequently examined using functional assays, for example, the assays described herein and those known in the art.

### Small Molecule Inhibitors

Small molecule inhibitors can be used as inhibitory agents (e.g., to inhibit and/or modulate the interaction between S100A8/S100A9 and RAGE or between S100A8/A9 and MDSCs) as disclosed herein. Any type of small molecule inhibitor that is known to one of skill in the art may be used. Many methods are known to identify small molecule inhibitors and commercial laboratories are available to screen for small molecule inhibitors. For example, chemicals can be obtained from the compound collection at Merck Research Laboratories (Rahway, N.J.) or a like company. For example, the compounds can be screened for inhibitory activity by automated robotic screening in a 96-well plate format. In summary, the compounds can be dissolved at an initial concentration of about 50 μM in DMSO and dispensed into the 96-well plate. For example, the 96-well plate assay may contain an appropriate number of units of S100A8/A9 and RAGE. Candidate small molecule inhibitors that cause greater than a 50% inhibition of binding can be further diluted and tested to establish the concentration necessary for a 50% inhibition of activity. The inhibitory effect of screened compound to disrupt S100A8/A9-RAGE binding can be monitored using, for example, an ELISA-type test with S100A8/A9 or RAGE immobilized on the surface. Binding assays can also be performed using any other assay known in the art used to screen protein-glycan interactions or protein-protein interactions (eg. yeast two hybrid systems, immunoprecipitation, immunocapture experiments coupled to enymatic or FACS detection etc.).

In some embodiments, the compositions and methods disclosed herein are used to treat diseased cells, tissues, organs, or pathological conditions and/or disease states in an animal (e.g., a mammalian subject including, but not limited to, humans and veterinary animals). In this regard, various diseases and pathologies are amenable to treatment or prophylaxis using the present methods and compositions. A non-limiting exemplary list of these diseases and conditions includes, but is not limited to, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like, T and B cell mediated autoimmune diseases; inflammatory diseases; infections; hyperproliferative diseases; AIDS; degenerative conditions, vascular diseases, and the like. In some embodiments, the cancer cells being treated are metastatic. In other embodiments, the cancer cells being treated are resistant to anticancer agents.

Many immune related diseases are known and have been extensively studied which are also amenable to treatment or prophylaxis using the present methods and compositions. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, etc.

The present methods and compositions are also amenable to the treatment of atopic and contact dermatitis, inflammatory bowel disease (IBD) such as ulcerative colitis and Crohn's disease, endotoxemia, arthritis, rheumatoid arthritis, psoriatic arthritis, adult respiratory disease (ARD), septic shock, multiple organ failure, inflammatory lung injury such as asthma, chronic obstructive pulmonary disease (COPD), airway hyper-responsiveness, chronic bronchitis, allergic asthma, bacterial pneumonia, psoriasis, eczema, helicobacter pylori infection, intraabdominal adhesions and/or abscesses as results of peritoneal inflammation (i.e. from infection, injury, etc.), systemic lupus erythematosus (SLE), multiple sclerosis, systemic sclerosis, nephrotic syndrome, organ allograft rejection, graft vs. host disease (GVHD), kidney, lung, heart, etc. transplant rejection, streptococcal cell wall (SCW)-induced arthritis, osteoarthritis, gingivitis/periodontitis, herpetic stromal keratitis, cancers including prostate, renal, colon, ovarian, cervical, leukemia, angiogenesis, restenosis and Kawasaki disease.

In some embodiments, infections suitable for treatment with the compositions and methods disclosed herein include, but are not limited to, infections caused by viruses, bacteria, fungi, mycoplasma, prions, and the like.

Some embodiments disclosed herein concern improved therapeutic approaches, wherein an effective amount of an inhibitory agent is combined or co-administered with at least one additional therapeutic agent (including, but not limited to, chemotherapeutic antineoplastics, apoptosis modulating agents, immunotherapeutics, antimicrobials, antivirals, antifungals, and anti-inflammatory agents) and/or therapeutic technique (e.g., surgical intervention, and/or radiotherapies).

A number of suitable anticancer agents are contemplated for combination or co-administration with an inhibitory agent of embodiments disclosed herein. Indeed, some embodiments contemplate, but are not limited to, administration of an inhibitory agent in combination or co-administered with numerous anticancer agents such as: agents that induce apoptosis; polynucleotides (e.g., anti-sense, ribozymes, siRNA); polypeptides (e.g., enzymes and antibodies); biological mimetics (e.g., gossypol or BH3 mimetics); agents that bind (e.g., oligomerize or complex) with S100A8/A9 and/or RAGE; alkaloids; alkylating agents; antitumor antibiotics; antimetabolites; hormones; platinum compouhds; monoclonal or polyclonal antibodies (e.g., antibodies conjugated with anticancer drugs, toxins, defensins), toxins; radionuclides; biological response modifiers (e.g., interferons (e.g., IFN-alpha.) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-κB modulators; anti-CDK compounds; HDAC inhibitors; and the like. Numerous other examples of chemotherapeutic compounds and anticancer therapies suitable for mixture or co-administration with the disclosed inhibitory agents are known to those skilled in the art.

In more embodiments, the inhibitory agents described herein and used in the methods disclosed are mixed or combined or co-administered with anticancer agents that induce or stimulate apoptosis. Agents that induce apoptosis which are suitable in such compositions, mixtures, therapies and methods include, but are not limited to, radiation (e.g., X-rays, gamma rays, UV); tumor necrosis factor (TNF)-related factors (e.g., TNF family receptor proteins, TNF family ligands, TRAIL, antibodies to TRAILR1 or TRAILR2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC®)); antisense molecules; antibodies (e.g., HERCEPTIN®, RITUXAN®, ZEVALIN®, and AVASTIN®); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON®, DELTASONE®, dexamethasone, dexamethasone intensol, DEXONE®, HEXADROL®, hydroxychloroquine, METICORTEN®, ORADEXON, ORASONE®, oxyphenbutazone, PEDIAPRED®, phenylbutazone, PLAQUENIL®, prednisolone, prednisone, PRELONE®, and TANDEARIL®); and cancer chemotherapeutic drugs (e.g., irinotecan (CAMPTOSAR®), CPT-11, fludarabine (FLUDARA®), dacarbazine (DTIC®), dexamethasone, mitoxantrone, MYLOTARG®, VP-16®, cisplatin, carboplatin, oxaliplatin, 5-FU®, doxorubicin, gemcitabine, bortezomib, gefitinib, bevacizumab, TAXOTERE® or TAXOL®); cellular signaling molecules; ceramides and cytokines; staurosporine, and the like.

In still other embodiments, compositions and methods described provide an inhibitory agent and at least one anti-hyperproliferative or antineoplastic agent selected from alkylating agents, antimetabolites, and natural products (e.g., herbs and other plant and/or animal derived compounds).

Alkylating agents suitable for use in the present compositions, mixtures, therapies, and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC®; dimethyltriazenoimid-azolecarboxamide).

In some embodiments, antimetabolites suitable for use in the present compositions, mixtures, therapies, and methods include, but are not limited to: 1) folic acid analogs (e.g., methotrexate (amethopterin)); 2) pyrimidine analogs (e.g., fluorouracil (5-fluorouracil; 5-FU®), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use with the compositions, mixtures, therapies, and methods described herein include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the compositions and methods disclosed herein. For example, the U.S. Food and Drug Administration maintains a formulary of oncolytic agents approved for use in the United States. International counterpart agencies to the U.S.F.D.A. maintain similar formularies.

In some embodiments, conventional anticancer agents for use in administration with the present inhibitory agents include, but are not limited to, adriamycin, 5-fluorouracil, etoposide, camptothecin, actinomycin D, mitomycin C, cisplatin, docetaxel, gemcitabine, carboplatin, oxaliplatin, bortezomib, gefitinib, and bevacizumab. These agents can be prepared and used singularly, in combined therapeutic compositions, in kits, or in combination with immunotherapeutic agents, and the like.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

In some embodiments, antimicrobial therapeutic agents may also be provided in addition to an inhibitory agent described herein. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

### Pharmaceutical Compositions/Formulations and Administration

Embodiments disclosed herein also relate to methods of administering an inhibitory agent to a subject in order to treat inflammation and/or cancer. The routes of administration can vary with the location and nature of the tumor, and include, e.g., intravascular, intradermal, transdermal, parenteral, intravenous, intramuscular, intranasal, subcutaneous, regional, percutaneous, intratracheal, intraperitoneal, intraarterial, intravesical, intratumoral, inhalation, perfusion, lavage, direct injection, and oral administration and formulation.

The term “intravascular” is understood to refer to delivery into the vasculature of a patient, meaning into, within, or in a vessel or vessels of the patient. In certain embodiments, the administration can be into a vessel considered to be a vein (intravenous), while in others administration can be into a vessel considered to be an artery. Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

Injection into the tumor vasculature is specifically contemplated for discrete, solid, accessible tumors. Local, regional or systemic administration also may be appropriate. For tumors of greater than about 4 cm, the volume to be administered can be about 4-10 ml (preferably 10 ml), while for tumors of less than about 4 cm, a volume of about 1-3 ml can be used (preferably 3 ml). Multiple injections delivered as single dose comprise about 0.1 to about 0.5 ml volumes. The inhibitory agent may advantageously be contacted by administering multiple injections to the tumor, spaced at approximately 1 cm intervals.

In the case of surgical intervention, an inhibitory agent can be administered preoperatively, to render an inoperable tumor subject to resection. Alternatively, an inhibitory agent can be administered at the time of surgery, and/or thereafter, to treat residual or metastatic disease. For example, a resected tumor bed may be injected or perfused with a formulation comprising an inhibitory agent that renders the inhibitory agent advantageous for treatment of tumors. The perfusion may be continued post-resection, for example, by leaving a catheter implanted at the site of the surgery. Periodic post-surgical treatment can be carried out.

Continuous administration also may be applied where appropriate, for example, where a tumor is excised and the tumor bed is treated to eliminate residual, microscopic disease. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well, and often depend on cancer type, cancer location, disease progression, and health and age of the patient. Certain types of cancer will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be resectable. Treatments with an inhibitory agent may increase the resectability of the tumor due to shrinkage at the margins or by elimination of certain particularly invasive portions. Following treatments, resection may be possible. Additional treatments subsequent to resection can serve to eliminate microscopic residual disease at the tumor site.

A typical course of treatment, for a primary tumor or a post-excision tumor bed, can involve multiple doses. Typical primary tumor treatment can involve a 6 dose application over a two-week period. The two-week regimen may be repeated one, two, three, four, five, six or more times. During a course of treatment, the need to complete the planned dosings may be re-evaluated.

Embodiments disclosed herein also relate to methods of reducing inflammation. Inflammation can be inhibited by administering an inhibitory agent disclosed herein. Tissues to be treated include, but are not limited to, gastrointestinal tissue (e.g., intestinal tissue), cardiac tissue, pulmonary tissue, dermal tissue, and hepatic tissue. For example, the tissue can be an epithelial tissue such as an intestinal epithelial tissue, pulmonary epithelial tissue, dermal tissue (i.e., skin), or liver epithelial tissue. Embodiments described herein relate to methods that lead to a reduction in the severity or the alleviation of one or more symptoms of an inflammatory disorder such as those described herein. Inflammatory disorders can be diagnosed and or monitored, typically by a physician using standard methodologies.

Inhibition of inflammation is typically characterized, for example, by a reduction of redness, pain and/or swelling of the treated tissue compared to a tissue that has not been contacted with an inhibitory agent. Tissues can be directly contacted with an inhibitory agent. Alternatively, the inhibitory agent can be administered systemically. Inhibitory agents are administered in an amount sufficient to decrease (e.g., inhibit) inflammation, e.g., by reducing an inflammatory response. An inflammatory response can be evaluated, for example, by morphologically by observing tissue damage, localized redness, and swelling of the affected area. Alternatively, an inflammatory response can be evaluated by measuring the amount of an inflammatory marker (e.g., a cytokine), for example, in the tissue or in the serum or plasma. Detection of inflammatory markers (e.g., cytokines) can be measured by methods known in the art. For example, cytokine production can be determined using an immunoassay. A decrease in white blood count can also indicate a decrease in inflammation.

Efficaciousness of treatment can be determined in association with any known method for diagnosing or treating the particular inflammatory disorder. Alleviation of one or more symptoms of the inflammatory disorder indicates that the compound confers a clinical benefit.

For example, gastrointestinal inflammatory disorders, including, for example, inflammatory bowel disease, Crohn's Disease, and colitis (i.e., ulcerative, ileitis or proctitis), can be treated with embodiments disclosed herein. Symptoms of gastrointestinal inflammatory disorder include, but are not limited to, abdominal pain, bloody diarrhea, fatigue, weight loss, loss of appetite, rectal bleeding and loss of body fluids and nutrients. Gastrointestinal inflammation can also cause problems such as arthritis, inflammation of the eye, liver disease (fatty liver, hepatitis, cirrhosis, and primary sclerosing cholangitis), osteoporosis, skin rashes, anemia, and kidney stones. Gastrointestinal inflammation can be diagnosed using a blood tests to check for anemia, which can indicate bleeding in the colon or rectum. In addition, a stool sample, can be taken to determine if there is bleeding or infection in the colon or rectum. Alternatively, a colonoscopy is performed to detect inflammation, bleeding, or ulcers on the colon wall. For the treatment of gastrointestinal inflammatory disorders, the inhibitory agent can be systemically administered or locally administered directly into gastric tissue. The systemic administration compound can be administered, for example, intravenously, rectally or orally. For local administration, a inhibitory agent-impregnated wafer or resorbable sponge can be placed in direct contact with gastric tissue. The inhibitory agent or mixture of compounds is slowly released in vivo by diffusion of the drug from the wafer and erosion of the polymer matrix.

The treatments may include various “unit doses.” Unit dose refers to a dose containing a predetermined-quantity of the therapeutic composition. The quantity to be administered, and the particular route and formulation, are within the skill of those in the clinical arts. A unit dose need not be administered as a single injection but may comprise continuous infusion over a set period of time. p In some embodiments disclosed herein, an inhibitory agent and one or more therapeutic agents, antibiotics, or anticancer agents are provided to an animal under one or more of the following conditions: at different periodicities, at different durations, at different concentrations, by different administration routes, etc. In some embodiments, the inhibitory agent is administered prior to the therapeutic or anticancer agent, e.g., 0.5, 1, 2 3, 4, 5, 10, 12, or 18 hours, 1, 2, 3, 4, 5, or 6 days, 1, 2, 3, or 4 weeks prior to the administration of the therapeutic or anticancer agent. In some embodiments, the inhibitory agent is administered after the therapeutic or anticancer agent, e.g., 0.5, 1, 2 3, 4, 5, 10, 12, or 18 hours, 1, 2, 3, 4, 5, or 6 days, 1, 2, 3, or 4 weeks after the administration of the anticancer agent. In some embodiments, the inhibitory agent and the therapeutic or anticancer agent are administered concurrently but on different schedules, e.g., the inhibitory agent is administered daily while the therapeutic or anticancer agent is administered once a week, once every two weeks, once every three weeks, or once every four weeks. In other embodiments, the inhibitory agent is administered once a week while the therapeutic or anticancer agent is administered daily, once a week, once every two weeks, once every three weeks, or once every four weeks.

### Injectable Compositions and Formulations

Injection of an inhibitory agent can be delivered by syringe or any other method used for injection of a solution, as long as the inhibitory agent can pass through the particular gauge of needle required for injection. A novel needleless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

### Diagnostic and Prognostic Applications

Some embodiments disclosed herein concern diagnostic and prognostic methods for the detection of inflammation and/or cancer (e.g., colon cancer). For example, the detection of the expression (or lack thereof) of a carboxylated glycan (e.g. expressed on RAGE) recognized by an antibody of embodiments disclosed herein (e.g., mAbGB3.1) provides a means of determining whether or not cells or tissue samples are cancerous. Carboxylated glycan levels may also be used to determine the sensitivity of certain cancer treatments. Such detection methods may be used, for example, for early diagnosis of the disease, to monitor the progress of the disease or the progress of treatment protocols, or to assess the grade of the cancer. The detection can occur in vitro or in vivo. Useful assays include, for example, an enzyme immune assay (EIA) such as enzyme-linked immunosorbent assay (ELISA), a radioimmune assay (RIA), a Western blot assay, an immunohistochemical assay, or a slot blot assay see, e.g., U.S. Pat. Nos. 4,366,241; 4,376,110; 4,517,288; and 4,837,168).

The detection of the expression profile of carboxylated glycans in cells may be carried out by any of several means well known to those of skill in the art. Some embodiments disclosed herein relate to methods of detecting carboxylated glycans that is immunological in nature. “Immunological” refers to the use of antibodies (e.g., polyclonal or monoclonal antibodies) specific for the carboxylated glycans (e.g. mAbGB3.1).

As used herein, the term “level” refers to expression levels of carboxylated glycans (e.g., the carboxylated glycan recognized by mAbGB3.1) and/or protein or to DNA copy number of an inflammation and/or cancer marker. Typically the level of the marker in a biological sample obtained from the subject is different (i.e., increased or decreased) from the level of the same marker in a similar sample obtained from a healthy individual (examples of biological samples are described herein).

As used herein, “predetermined level” refers to the level of expression of an inflammation and/or cancer marker (e.g., carboxylated glycan expressed on RAGE) in normal, non-cancerous tissue. In some embodiments, cancer (e.g., breast cancer or lung cancer) can be diagnosed by assessing whether carboxylated glycan expression varies from a predetermined level by greater than or equal to 50%, 55%, 60%, 65%, 70%, 75%, 80%, 85%, 90%, 91%, 92%, 93%, 94%, 95%, 96%, 97%, 98%, 99% or 100%.

Numerous well known tissue or fluid collection methods can be utilized to collect the biological sample from the subject in order to determine the level of carboxylated glycan, DNA, RNA and/or polypeptide of the variant of interest in the subject. Examples include, but are not limited to, fine needle biopsy, needle biopsy, core needle biopsy and surgical biopsy (e.g., brain biopsy), lavage, and any known method in the art. Regardless of the procedure employed, once a biopsy/sample is obtained the level of the marker can be determined and a diagnosis can thus be made. For example, tissue sample may be obtained by biopsy. The sample of cells or tissue can then be prepared and exposed to an antibody or a mixture of antibodies according to means which are known to those of skill in the art. Samples can then be prepared for immunohistochemical analysis of gene expression, for example, using tissue microarrays.

Other means of detecting the expression profile of inflammation and/or cancer markers include but are not limited to, for example, detection of mRNA encoding the protein. Those of skill in the art are well acquainted with methods of mRNA detection, e.g., via the use of complementary hybridizing primers (e.g., labeled with radioactivity or fluorescent dyes) with or without polymerase chain reaction (PCR) amplification of the detected products, followed by visualization of the detected mRNA via, for example, electrophoresis (e.g., gel or capillary); by mass spectroscopy; etc. Any means of detecting the presence of the mRNA in an amount lower than normal or baseline control (or to detect the absence of the mRNA. For example, an immunoassay can measure the level of gene expression or activity by measuring the level of mRNA. The level of mRNA may also be measured, for example, using ethidium bromide staining of a standard RNA gel, Northern blotting, primer extension, or nuclease protection assay.

Other immune assays which may be utilized include, but are not limited to agglutination methods, precipitation methods, immunodiffusion methods, immunoelectrophoresis methods, nephelometry, gel electrophoresis followed by Western blot, dot blots, affinity chromatography, immune-fluorescence, and the like. In addition, other detection methods known to those of skill in the art may be used, such as gas chromatography/mass spectrometry, HPLC, and gel electrophoresis followed by sequencing.

Other methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer, and is hereby incorporated by reference in its entirety.

### Monitoring Therapy

The phrase “monitoring therapy” refers to determining the relative amount of inflammation and/or cancer cells in the body of a patient before, during and/or after therapy.

Some embodiments disclosed herein relate to methods for monitoring the progress or efficacy of therapy in a subject. Subjects identified as having inflammation and/or cancer and undergoing cancer therapy can be administered labeled antibodies (e.g. labeled antibodies against a carboxylated glycan expressed on RAGE (e.g., mAbGB3.1).

Subjects can be administered a labeled antibody before the onset of treatment or during treatment. Cells containing the label can be assayed for and this measurement can be compared to one obtained at a subsequent time during the therapy and/or after therapy has been completed. In this way, it is possible to evaluate the inhibition of inflammation and/or cancer cell proliferation, and the effectiveness of the therapy. For example, in instances when only living cancer cells will be detected via the labeled antibody, the therapy can continue until a minimal amount of label is detected.

Some embodiments disclosed herein also relate to methods for determining the amount of cancer cells present in a subject. By detecting the label or detecting the absence of the label, one can determine whether cancer cells are present within the subject and the amount of label measured may be proportional or inversely related to the amount of cancer cells present in the subject.

### Kits

Some embodiments disclosed herein provide for a kit for detecting cancer comprising an inhibitory agent and instructions for use.

In one embodiment, the kit may comprise a reference sample, e.g., a negative and/or positive control. In that embodiment, the negative control would be indicative of a normal cell type and the positive control would be indicative of cancer. Such a kit may also be used for identifying potential candidate therapeutic agents for treating cancer. In one embodiment, the first binding moiety is labeled. In one embodiment, the kit further comprises a second binding moiety which binds specifically to the first binding moiety.

The above mentioned kit can be used for the detection of any cell-proliferative cancer including, without limitation, breast cancer, ovarian cervical cancer, prostate cancer, colon cancer, lung cancer, skin cancer, leukemia, lymphoma, melanoma or any other type of cancer. The kit may also be used to determine the aggressiveness or grade of cancer.

In one embodiment, the inhibitory agent in the kit can be an antibody or fragment thereof which specifically binds to a carboxylated glycan. Antibodies and binding fragments thereof can be lyophilized or in solution. Additionally, the preparations can contain stabilizers to increase the shelf-life of the kits, e.g., bovine serum albumin (BSA). Wherein the antibodies and antigen binding fragments thereof are lyophilized, the kit can contain further preparations of solutions to reconstitute the preparations. Acceptable solutions are well known in the art, e.g., PBS. In one embodiment, the antibody is a polyclonal antibody, a monoclonal antibody, a humanized antibody, a chimeric antibody, a recombinant antibody, or fragment thereof.

In some embodiments, the kits can further include the components for an immunohistochemical assay for measuring one or more markers (e.g. carboxylated glycans expressed on RAGE). Samples to be tested in this application include, for example, blood, serum, plasma, urine, lymph, tissue and products thereof.

Alternatively, the kits can be used in immunoassays, such as immunohistochemistry to test subject tissue biopsy sections. The kits may also be used to detect the presence of a marker in a biological sample obtained from a subject using immunohistocytochemistry.

The compositions of the kit can be formulated in single or multiple units for either a single test or multiple tests.

Embodiments disclosed herein further provide for a kit for use in, for example, the screening, diagnosis or monitoring of inflammation and/or cancer or inflammation treatment and/or cancer treatment. Such a kit may comprise an antibody (e.g. an antibody that recognizes a carboxylated glycan expressed on RAGE), a reaction container, various buffers, secondary antibodies, directions for use, and the like. In these kits, antibodies may be provided with means for binding to detectable marker moieties or substrate surfaces. Alternatively, the kits may include antibodies already bound to marker moieties or substrates. The kits may further include positive and/or negative control reagents as well as other reagents for carrying out diagnostic techniques. For example, kits containing antibody bound to multiwell microtiter plates can be provided. The kit may include a standard or multiple standard solutions containing a known concentration of a marker or other proteins for calibration of the assays. A large number of control samples are assayed to establish the threshold, mode and width of the distribution of marker in normal cells and tissues against which test samples are compared. These data is provided to users of the kit.

The following examples provide illustrations of some of the embodiments described herein but are not intended to limit invention.

Examples 1-7 described below demonstrate that carboxylated glycans, RAGE, and carboxylated glycan-binding proteins S100A8/A9, exert tumorigenic functions in the setting of inflammation. In addition, RAGE signaling promotes the development of CAC. Embodiments of the invention relate to the discovery that carboxylated glycans mediated S100A8/A9 and RAGE binding and that these glycans promoted receptor mediated signaling leading to the pathogenesis of CAC.

### EXAMPLE 1

### Carboxylated Glycans, RAGE and S100A8/A9 are Expressed in Colorectal Tumors

Carboxylated glycans are expressed on endothelial cells and macrophages in normal human colon and on inflammatory infiltrates in colon tissues from patients with colitis (Srikrishna et al. (2005) J Immunol, 175, 5412-22). To determine whether carboxylated glycans, RAGE and S100A8/A9 are also expressed in colon tumors, immunohistochemistry of human colorectal tumor tissues was performed. Carboxylated glycans, as seen by staining with mAbGB3.1, and RAGE were expressed on endothelial cells and macrophages in almost all the colon tumor tissues and paired adjacent normal tissues. Staining of tumor vasculature by antibody mAbGB3.1 was more intense in a few tumor samples. In addition, in one moderately differentiated colon carcinoma (stage IIIB), there was staining of tumor epithelial cells by both mAbGB3.1 and anti-RAGE antibodies, while adjacent normal epithelial cells were negative. Few S100A9 positive macrophages were present in normal colon. However, positive cells were found within tumor stroma in all tumor tissues examined. Cells were also positive for S100A8. Increased expression of RAGE on colon tumor epithelial cells and of S100A8/A9 in tumor stroma has also been reported earlier (Harada et al. (2007) Int J Cancer, 121, 1072-8; Stulik et al. (1999) Electrophoresis, 20, 1047-54; Sickert et al. (2007) Anticancer Res, 27, 1693-700).

### EXAMPLE 2

### Carboxylated Glycans are Expressed on a Subpopulation of RAGE Molecules in Tumor Cells

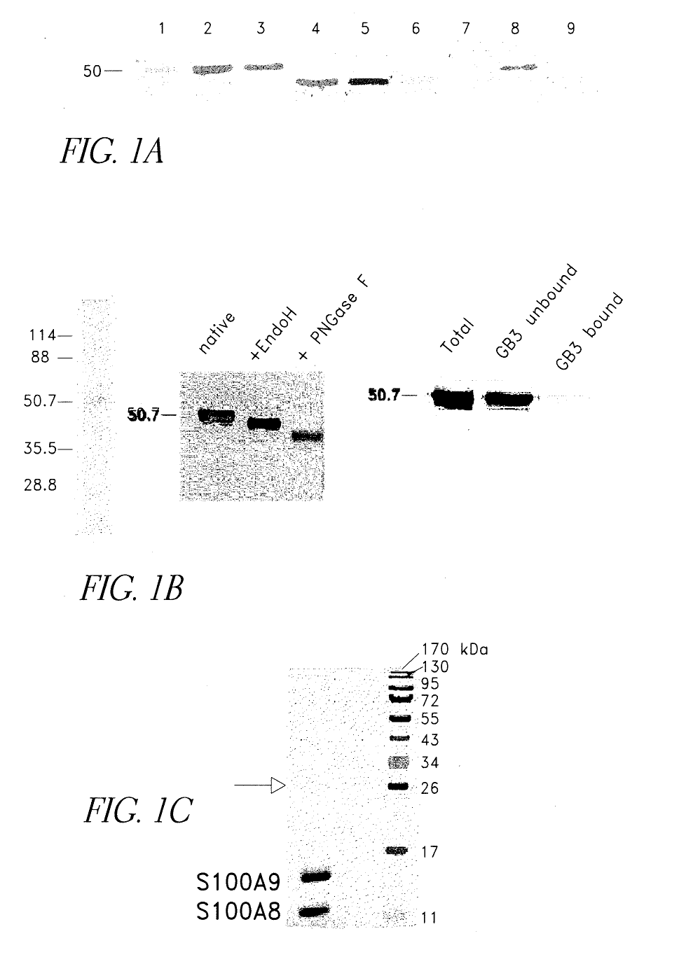

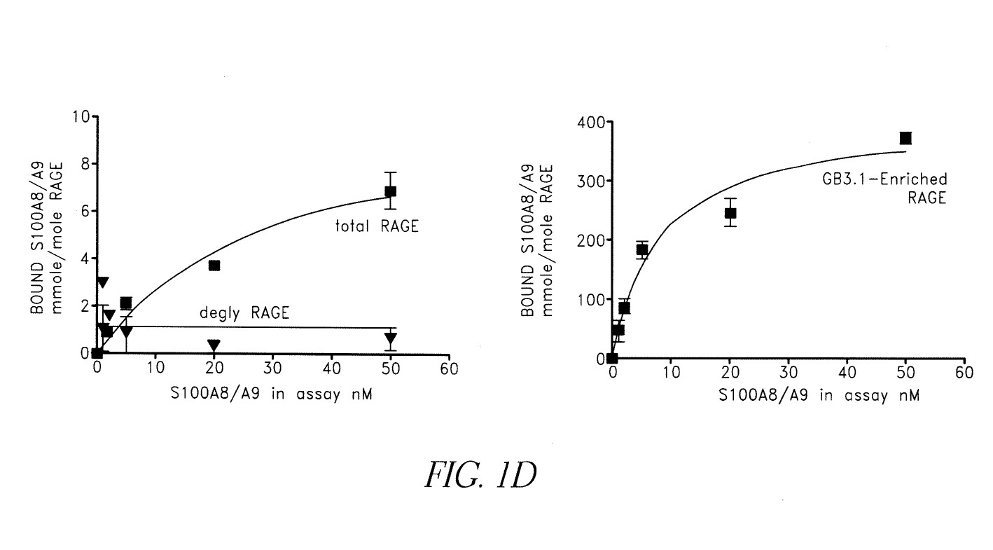

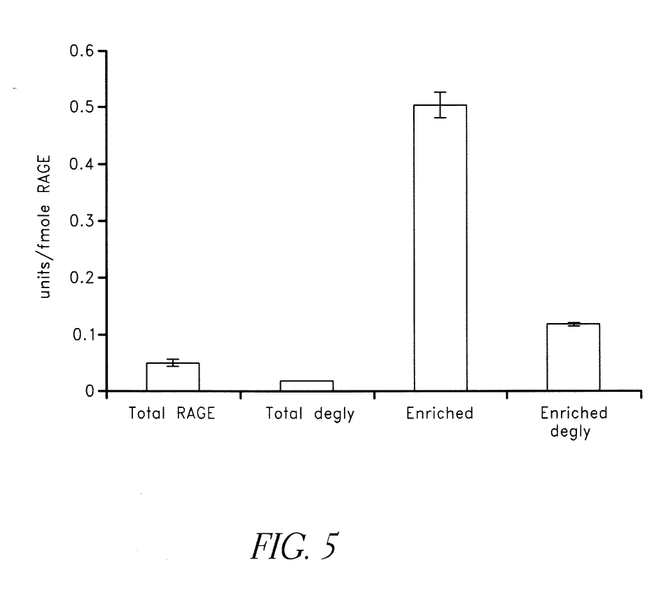

To examine whether RAGE expressed on tumor cells is modified by carboxylated glycans, membrane preparations from cultured colon tumor cells were analyzed. RAGE is expressed on mouse and human colon tumor cells and is glycosylated as evident from a band shift upon PNGase F deglycosylation (FIG. 1A). Cell surface expression of RAGE and carboxylated glycans on tumor cells was confirmed by flow cytometry. Less than 2% of RAGE from colon tumor cells was immunoprecipitated by mAbGB3.1, suggesting that RAGE from tumor cells could be modified by carboxylated glycans (FIG. 1A). Since yields of RAGE from tumor cells were low, to further confirm whether only a subpopulation of RAGE molecules is modified by carboxylated glycans, RAGE was purified to >98% homogeneity from bovine lung, a rich source of the protein. Homogeneity was confirmed by Coomassie and silver staining (FIG. 1B). Treatment with EndoH and PNGaseF showed that both N-glycosylation sites on RAGE were occupied, by EndoH sensitive glycan chains and by EndoH-resistant, PNGaseF-sensitive chains (FIG. 1B). When purified RAGE was incubated with mAbGB3.1, a majority of RAGE remained unbound, even after two rounds of incubation with mAbGB3.1 immobilized beads. Only 5% of total RAGE bound to mAbGB3.1 and could be eluted by high pH or by carboxylated glycopeptides (FIG. 1B). This enriched fraction of RAGE showed >10 fold increase in mAbGB3.1 reactivity compared to total RAGE. Most of the binding was lost upon deglycosylation suggesting that it was glycan-dependent (FIG. 5). Soluble human RAGE was also expressed in HeLa cells and found that 1-2% of RAGE express carboxylated glycans. These findings confirmed that carboxylated glycans are expressed on a subpopulation of RAGE molecules.

### EXAMPLE 3

### S100A8/A9 Complex Binds to a Subpopulation of RAGE Expressing Carboxylated Glyeans

RAGE binds many S100 family proteins including S100A12, S100A1, S100B and S100P and the interactions lead to intracellular signaling (Hofmann et al. (1999) Cell, 97, 889-901; Huttunen et al. (2000) Journal of Biological Chemistry, 275, 40096-40105; Fuentes et al. (2007) Dis Colon Rectum, 50, 1230-40). To determine if S100A8/A9 directly bound RAGE and to examine the role of carboxylated glycans in binding, increasing amounts of purified mouse S100A8/A9 (FIG. 1C) were incubated with 1) immobilized total RAGE, 2) RAGE deglycosylated with PNGaseF under non-denaturing conditions that removed both N-glycans, and 3) mAbGB3.1 enriched RAGE. Purified total RAGE binds S100A8/A9 with a K_{d} of approximately 34.4±13 nM, and a Bmax (maximum binding sites) of 11.4±2.2 mmoles/mole RAGE (binding potential (Bmax/Kd) of 0.36±0.07). Deglycosylation almost completely abolished binding (FIG. 1D). This shows that only a very small fraction (approximately 1%) of the RAGE molecules carry S100A8/A9 binding sites and that N-glycans contribute significantly to binding. In support of this, the subpopulation of RAGE enriched for carboxylated glycans by mAbGB3.1 showed higher affinity interaction with a K_{d} of 7.62±1.83 nM and a Bmax of 402.1±30.5 mmoles/mole RAGE (FIG. 1D). This is a 35-fold increase in the molar binding and >100 fold increase in binding potential (Bmax/Kd of 55±9.2), suggesting that carboxylated glycans form critical binding sites for S100A8/A9 on RAGE.

### EXAMPLE 4

### S100A8/A9 Binds to Colonic Tumor Cells in a Carboxylated Glycan-Dependent Manner Promoting Intracellular Activation of NF-κB and Cell Proliferation

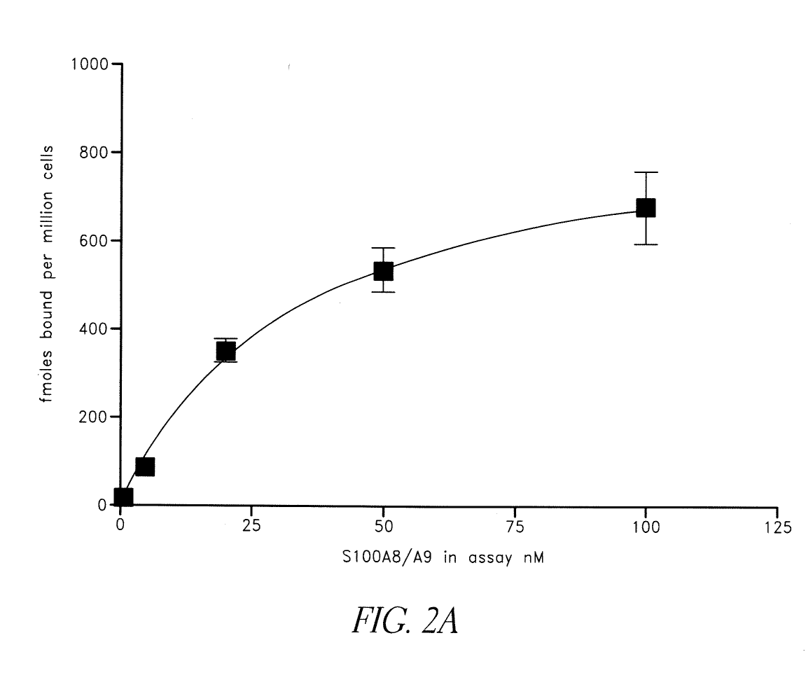

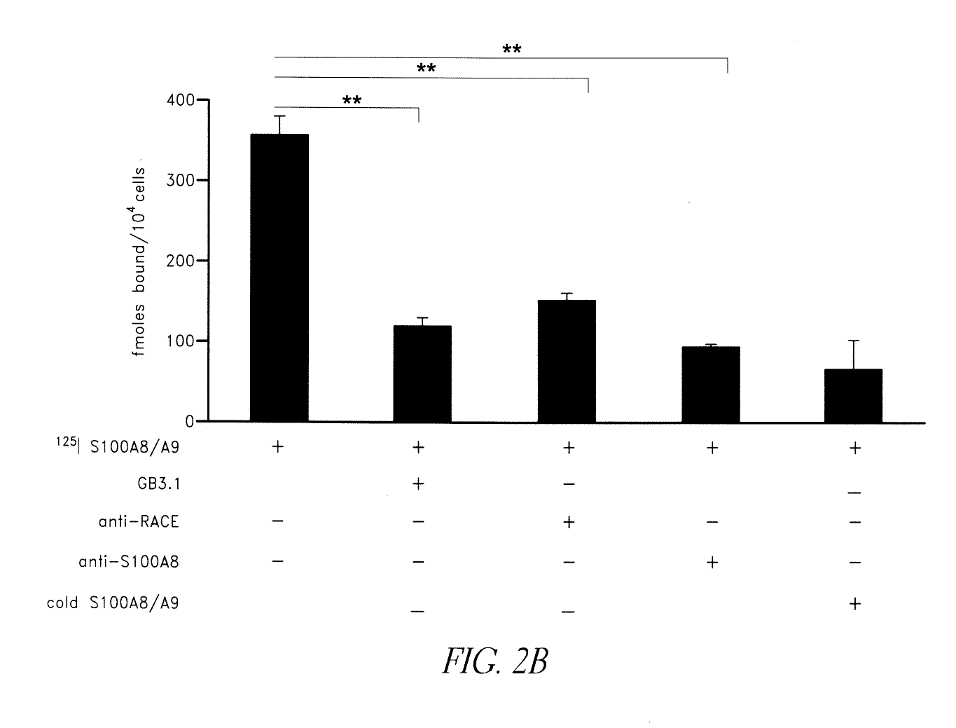

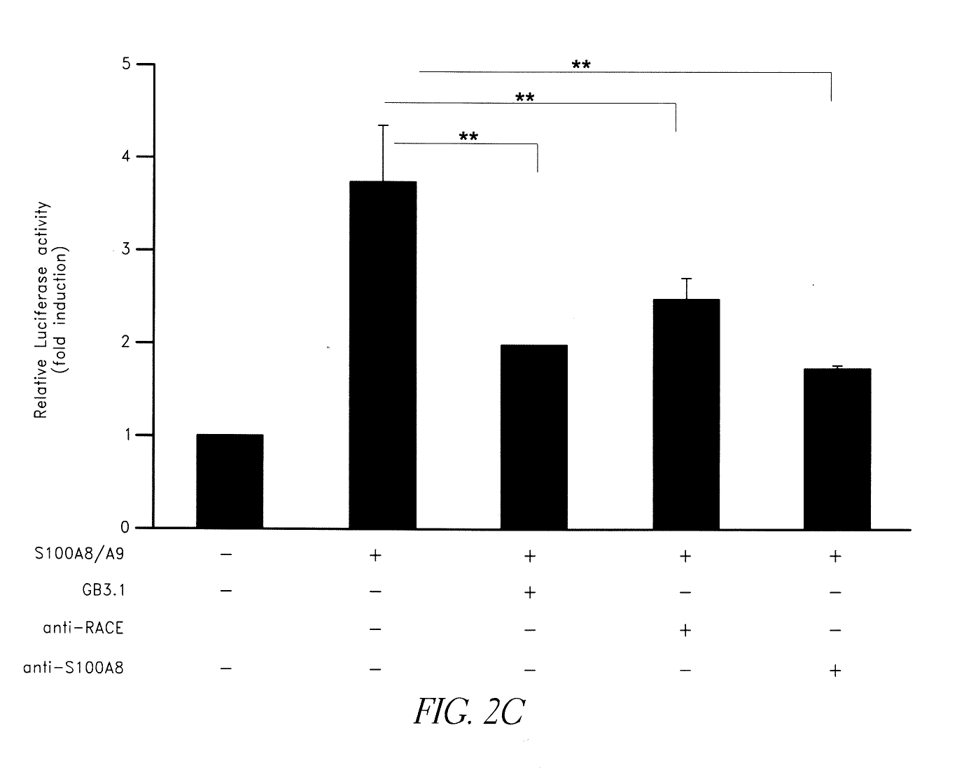

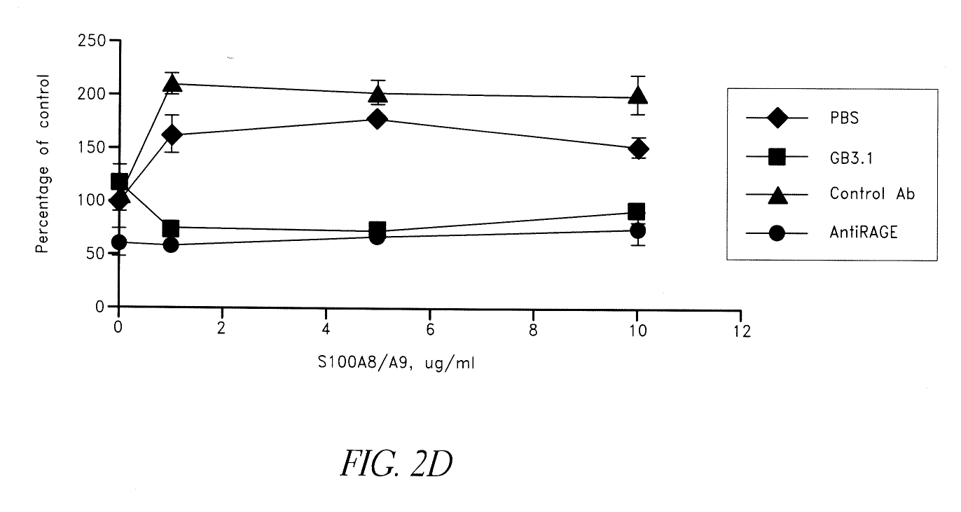

S100A8/A9 proteins are secreted in response to stimuli and have extracellular effects. To examine if S100A8/A9 binding sites are present on colon tumor cells, a radio-ligand binding assay was performed using ¹²⁵I-labeled purified mouse S100A8/A9. CT-26 tumor cells showed specific saturable binding sites with a Kd of 35.09±7.45 nM and a Bmax of 0.920±0.077 pmoles/million cells (FIG. 2A). ¹²⁵I S100A8/A9 binding was displaced by 50-fold molar excess of cold ligand or 10-fold molar excess of anti-S100A8 (FIG. 2B). To examine if the binding involves interaction with glycans on RAGE, cells were incubated with ¹²⁵I S100A8/A9 in the presence of mAbGB3.1 or anti-mouse RAGE. Binding was significantly reduced in the presence of mAbGB3.1 and anti-RAGE showing that RAGE and carboxylated glycans contribute to S100A8/A9 binding on tumor cells (FIG. 2B).

Ligand binding to RAGE mediates downstream signaling events in cells leading to NF-κB activation. Therefore S100A8/A9 induced NF-κB activation in colon tumor cells was studied using transient transfection with a luciferase expression plasmid containing four tandem repeats of NF-κB binding site and R. reniformis luciferase expression construct as an internal control. The transfected cells were treated with endotoxin free S100A8/A9. At low concentrations (0.5 ug/ml, approx 20 nM) S100A8/A9 stimulated NF-κB-dependent transcription of luiferase within the cells. Preincubation with mAbGB3.1 or anti-RAGE prior to stimulation decreased NF-κB expression (FIG. 2C) while an isotype control antibody had no or minimal effect.

Since NF-κB activation plays an important role in intestinal cell survival and homeostasis (Jobin et al. (2000) Am J Physiol Cell Physiol, 278, C451-62), whether S100A8/A9 promote colon tumor cell proliferation was next investigated. Cells were untreated or treated with S100A8/A9 in low serum medium in the presence or absence of inhibitors and cell proliferation was assayed using MTS reagent. At lower concentrations (1 μg/ml ˜40 nM), S100A8/A9 induced significant cell growth (FIG. 2D). Cell proliferation however did not increase with increasing concentrations of S100A8/A9 and only moderately with increasing time, similar to the effects of S100A8/A9 on other human tumor cells (Ghavami et al. (2008) J Leukoc Biol. In Press). This suggests that S100A8/A9 may be rapidly internalized or degraded. mAbGB3.1 and anti-RAGE reduced S100A8/A9 induced early cellular proliferation (FIG. 2D). Anti-RAGE also reduced cell proliferation in untreated cells, while mAbGB3.1 had no effect, suggesting that RAGE is important for tumor cell growth even in the absence of S100A8/A9, and S100A8/A9 induced cell proliferation may depend on carboxylated glycans expressed on RAGE and/or other proteins.

### EXAMPLE 5

### Myeloid Progenitor Cells Expressing S100A8/A9 Infiltrate Colon Tumors

To further understand the role of S100A8/A9, RAGE and carboxylated glycans in inflammation induced tumorigenesis, the effects of anti-carboxylated glycan antibody administration in a mouse model of CAC was tested. Inflammation-induced colon carcinogenesis can be modeled in mice by injection of the pro-carcinogen AOM followed by a single or multiple exposures to DSS. DSS causes epithelial damage and activates macrophages inducing an acute colonic inflammation. This initial response progresses to chronic inflammation over time when adaptive immune system responses are triggered. Animals develop chronic inflammation, colonic dysplasia and adenoma within 12-20 weeks of combined administration of AOM and single or multiple cycles of DSS (Greten et al. (2004) Cell, 118, 285-96; Tanaka et al. (2003) Cancer Sci, 94, 965-73; Dieleman et al. (1998) Clin Exp Immunol, 114, 385-91; Melgar et al. (2005) Am J Physiol Gastrointest Liver Physiol).

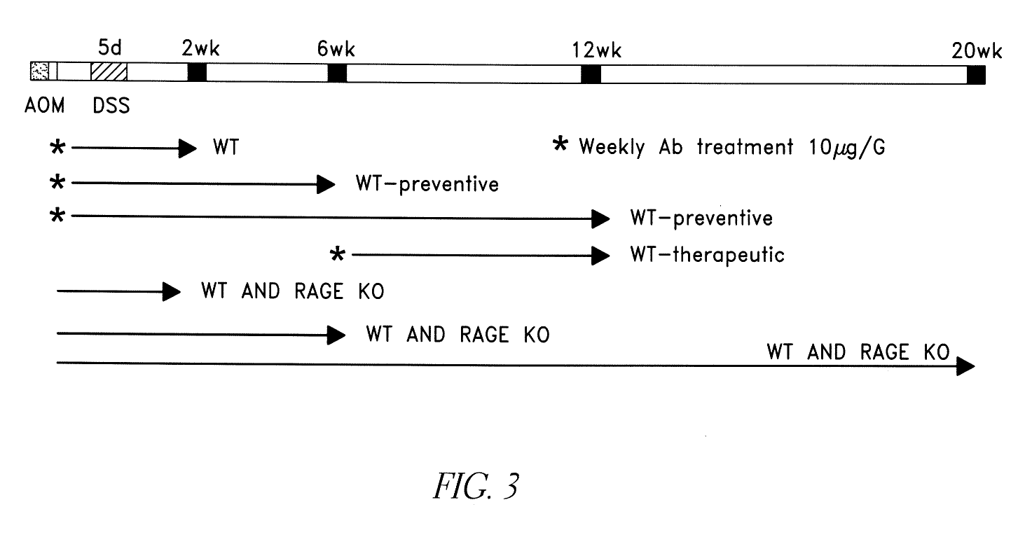

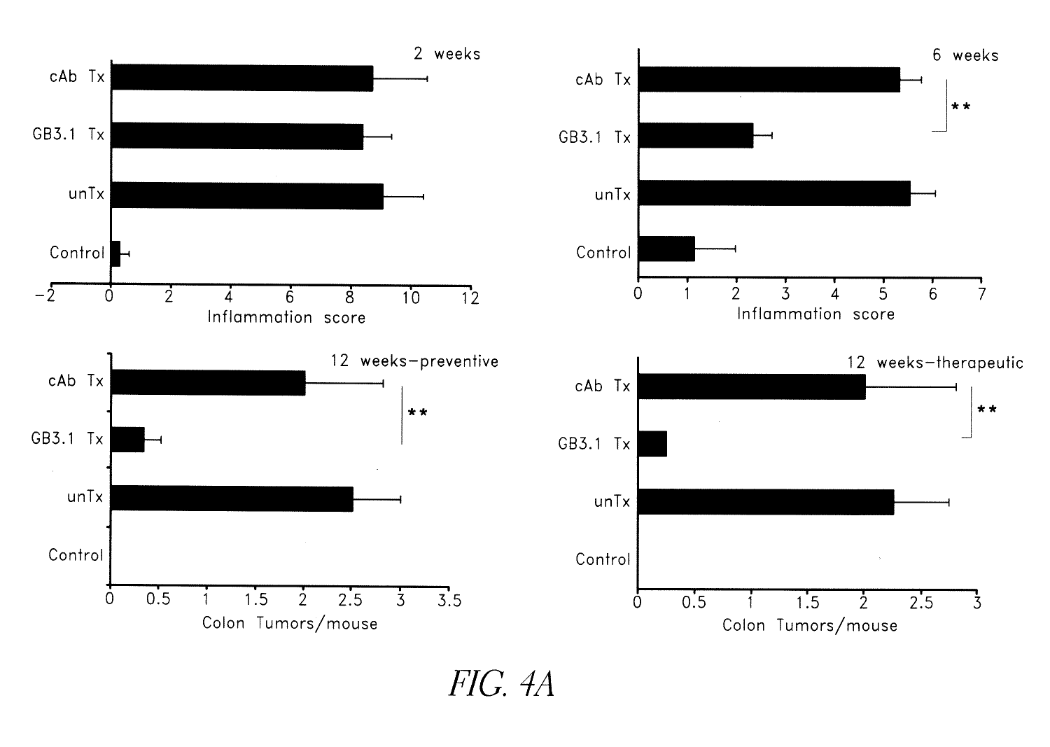

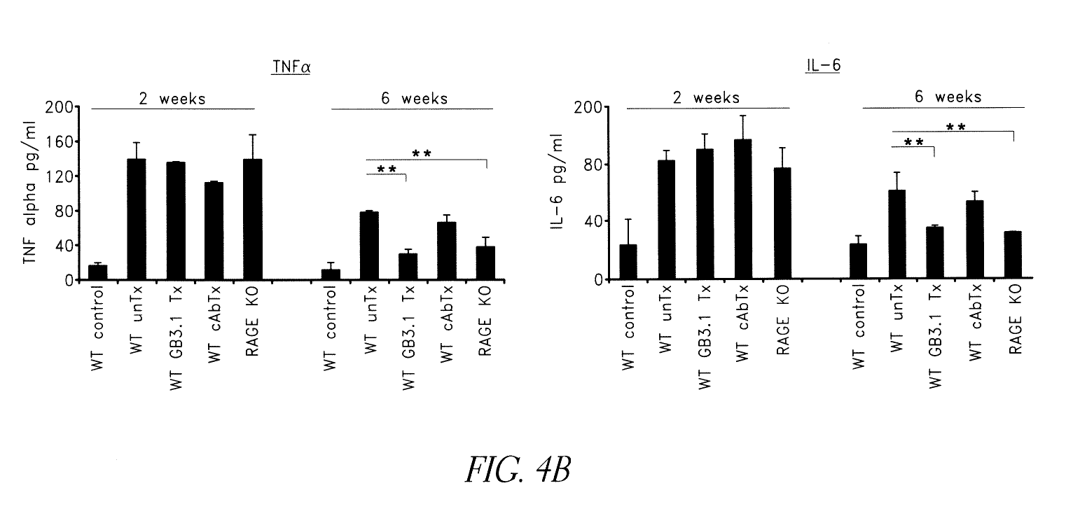

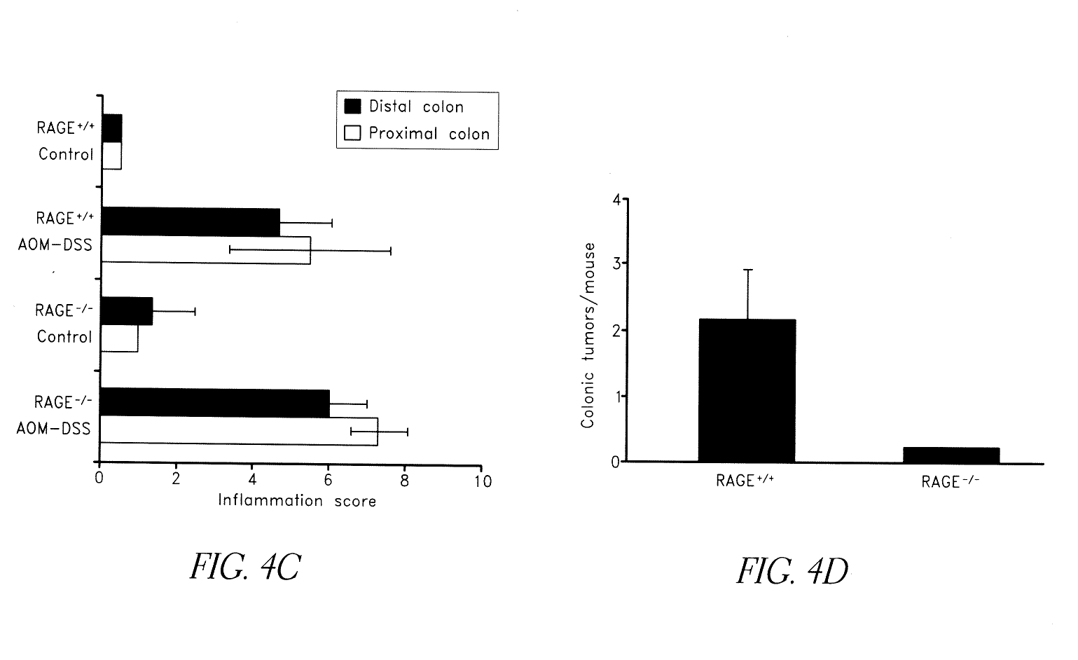

CAC was induced in mice using a single low dose of AOM followed by a single week of treatment with DSS (FIG. 3, ref (Suzuki et al. (2004) Cancer Sci, 95, 721-7; Tanaka et al. (2003) Cancer Sci, 94, 965-73). The animals exhibited weight loss and diarrhea during the acute phase that resolved within 1-2 wks after DSS treatment. Six wks after DSS, there were no clinical symptoms except for occasional soft stools. By 12 to 20 weeks, a few mice developed mild rectal prolapse and bloody stools. Histologically, there was significant colonic inflammation 2 weeks after DSS (FIG. 4A). Colonic inflammation was appreciable at 6 weeks even though it was less severe than at 2 weeks (FIG. 4A). In addition, low-grade dysplasia was evident at 6 weeks after initiation of disease, and by 12 weeks all the mice developed high-grade dysplasias and adenomas (2-3 tumors per mouse, 100% penetrance). By 20 weeks, tumors were macroscopically visible. Serum levels of NF-κB dependent gene products TNFα, and IL-6 were elevated two weeks after DSS, and remained higher than normal six weeks after DSS.

Diffuse staining for S100A8 and S100A9 was observed in the colons 2 weeks after initiation. Expression of these proteins is restricted to neutrophils and immature macrophages or monocytes in early stage differentiation (Roth et al. (1993) Biochem Biophys Res Commun, 191, 565-70) and they are absent on mature macrophages (Odink et al. (1987) Nature, 330, 80-2). In addition, by 12 to 20 wks of disease initiation, infiltrating cells positive for S100A8/A9 were found in all regions of dysplasia and adenoma, similar to infiltration seen in human colon tumors by immunohistochemistry as described above, but they were absent in adjacent regions of no disease activity from the same colons. The cells positive for S100A8/A9 were F4/80 negative, suggesting that they were not resident macrophages.

To further understand the phenotypic nature of S100A8/A9 positive cells in tumors, colons were examined for CD11b⁺ (monocyte), Gr1⁺ (neutrophil) and CD11b⁺/Gr1⁺ (myeloid progenitor) cells. A few CD11b⁺ cells were found in normal lamina propria, but Gr1⁺ cells and double positive cells were absent. However, CD11b⁺, Gr1⁺ single positive and CD11b⁺/Gr1⁺ double positive cells were present in larger numbers in regions of dysplasia and tumors. Immature myeloid progenitor cells are frequently found in tumors in mice and in many cancer patients (Sinha et al. (2005) Cancer Immunol Immunother, 54, 1137-42; Serafini et al. (2006) Semin Cancer Biol, 16, 53-65; Nagaraj et al. (2007) Adv Exp Med Biol, 601, 213-23). These cells, identified phenotypically in mice as Gr1⁺CD11b⁺ cells, and known as myeloid derived suppressor cells (MDSC) induce immune suppression against tumor antigens. MDSC are also found in inflammation induced skin tumors of wild type mice but not in RAGE^{-/-} tumors (Gebhardt et al. (2008) J Exp Med, 205, 275-85). MDSC from mice with mammary tumors showed up-regulation of intracellular S100A8/A9 and secreted them in response to stimuli (Sinha P et al., submitted). It is therefore likely that the S100A8/A9 positive cells in regions of colonic dysplasia and adenoma are immature myeloid progenitor cells adding to the heterogeneity of leukocyte populations within the tumor microenvironment.

### EXAMPLE 6

### Administration of Anti-Carboxylated Glycan Antibody Reduces Chronic Inflammation and Tumorigenesis

To investigate a functional role of the glycans in this model, separate groups of AOM/DSS mice were treated with mAbGB3.1 or isotype control antibody. mAbGB3.1 did not block the initial DSS induced injury (2 weeks after DSS, FIG. 4A), and treated mice did not show any difference in weight loss, clinical signs of inflammation or levels of pro-inflammatory cytokines in serum (FIG. 4B), suggesting that the glycans may not play a role in the initial innate immune responses to DSS. However, administration of mAbGB3.1 reduced inflammation at 6 wks and the incidence of tumors by about 75% at 12 weeks after initiation (FIG. 4A). In contrast the control antibody treated mice showed only a minimal decrease (20%) in incidence of inflammation and dysplasia. Levels of TNFα and IL-6 in serum were also reduced in mAbGB3.1 treated mice at 6 wks and 12 wks after initiation of disease (FIG. 4B). These results suggest that reduction in tumor incidence in mAbGB3.1 treated mice is not due to block in initial acute colitis but to block in transition to chronic inflammation and subsequent tumorigenesis. To establish whether the glycans independently promoted inflammation-mediated tumorigenesis, the effect of antibody administration was examined after establishment of chronic colitis. In fact, mAbGB3.1 treatment started 6 wks after initiation of disease significantly reduced the incidence of dysplasia and adenoma (FIG. 4A), suggesting that the glycans play dual roles in CAC, both in adaptive immune responses leading to chronic inflammation, and in inflammation mediated tumorigenesis.

### EXAMPLE 7

### RAGE Deficient Mice are Resistant to the Onset of CAC

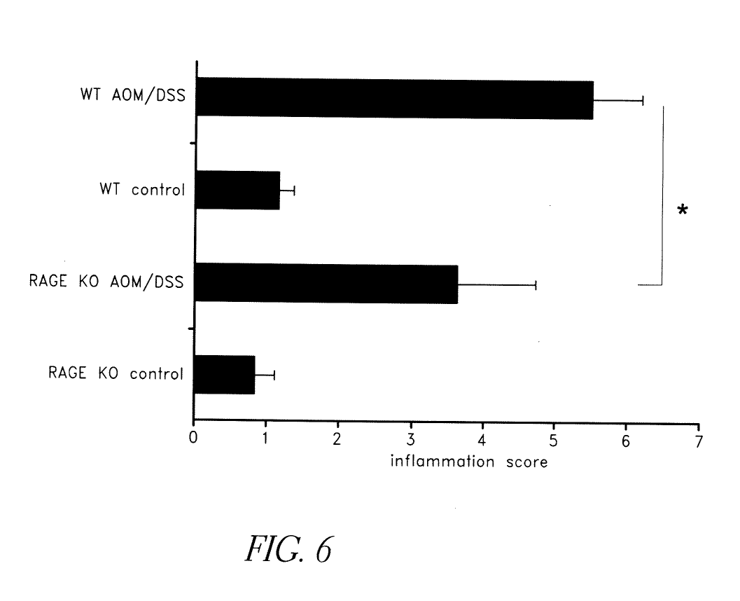

To evaluate the role of RAGE in S100A8/A9 and glycan mediated signaling events leading to CAC, the AOM/DSS protocol was applied in RAGE^{-/-} mice and wild type mice. Both RAGE^{-/-} and age matched C57BL/6 wild type mice lost up to 10% of body weight after DSS treatment before recovery. Proximal and distal colons showed evidence of inflammation in both RAGE^{-/-} mice and wild type mice (FIG. 4C) and serum TNFα and IL-6 levels were elevated in both groups (FIG. 4B), demonstrating that RAGE does not play a role in the initial acute inflammatory events triggered by DSS. However, serum levels of the pro-inflammatory cytokines were reduced in the RAGE^{-/-} mice at 6 wks after disease initiation compared to wild type mice (FIG. 4B) and colonic inflammation was moderately reduced (FIG. 6). In addition, there was a dramatic reduction in tumor incidence in RAGE^{-/-} mice 20 wks after AOM/DSS. Few dysplatic lesions found in RAGE^{-/-} mice were small and low grade. In contrast, all the wild type mice developed adenomas (1-3 tumors per mouse), with a few early invasive adenocarcinomas by 20 wks. These findings show that RAGE is essential for the pathogenesis of CAC, complementing a recently published study on RAGE signaling in skin carcinogenesis (Gebhardt et al. (2008) J Exp Med, 205, 275-85), and highlighting the importance of RAGE in inflammation mediated cancers.

Thus, the studies showedcarboxylated glycans expressed on a subpopulation of RAGE on tumor cells provide critical binding sites for S100A8/A9. The glycans and RAGE also mediate S100A8/A9 induced downstream signaling in tumor cells and cell proliferation. Using a colitis-induced cancer model that involves an acute inflammation phase, a chronic inflammation phase and a tumorigenesis phase, the glycans and RAGE were shown to be important both in the chronic inflammation and tumorigenesis phases. S100A8/A9 positive cells were found both in inflamed tissues and within tumor stroma. These findings demonstrate that the glycans, RAGE and S100A8/A9 are important in both facets of inflammation-based cancers.

In summary, the findings described in Examples 1-7 show that RAGE, S100A8/A9 and carboxylated glycans form important components of epithelial and stromal cells promoting molecular interactions leading to CAC. This complex signaling pathway represents a potential target for pharmacological interventions.

The following section describes in greater detail some of the materials and methods used in Examples 1-7.

### Human Tissues

Paired tumor and normal adjacent colon tissue samples (n=9) snap frozen in liquid nitrogen after surgery were provided by the Cooperative Human Tissue Network (CHTN, National Cancer Institute). Samples were from both male and female patients in the age range from 52 to 80 years. Pathology reports were provided by CHTN for each tissue sample, and included well-differentiated, moderately differentiated and poorly differentiated adenocarcinomas (AJCC staging: 2 cases of stage I, 1 case of stage IIA, 1 case of stage IIB, 1 case of stage IIIB, 3 cases of stage IIIC and 1 case of stage IV). 5 μm cryosections were made and stored frozen until analysis.

### Mice

RAGE^{-/-} mice were generated as described (Liliensiek et al. (2004) J Clin Invest, 113, 1641-50). They were backcrossed to C57BL/6 mice for more than 10 generations. 6-8 week old RAGE^{-/-} mice, RAGE^{+/+} littermates or age-matched wild type mice were used for experiments. All animal protocols were approved by the Burnham Institute for Medical Research Institutional Animal Care and Use Committee and were in compliance with NIH policies.

### Tumor Induction

CAC was induced in mice using AOM and single cycle of DSS as described (Suzuki et al. (2004) Cancer Sci, 95, 721-7). Animals were constantly monitored for clinical signs of illness, and were sacrificed at the end of 2 wks, 6 wks, 12 wks or 20 wks after DSS. Blood samples were collected by retro-orbital bleeding prior to induction of disease and at time points as above. In the preventive protocol of antibody treatment, mAbGB3.1 (10 μg/g) was administered i.v. in 100 μl PBS once a week, until 6 wks or 12 wks of disease initiation. For every time point, separate groups of control mice were either left untreated or administered an equivalent amount of a non-specific isotype control antibody. In the therapeutic protocol, mice received the antibodies weekly starting 6 wks after initiation of disease and sacrificed at 12 wks. Separate groups of RAGE^{-/-} mice or age matched control mice were subjected to the same AOM-DSS protocol and sacrificed at 2, 6 or 20 wks after disease initiation. At each time point, colons were excised, fixed as “Swiss-rolls” in 4% buffered formalin and embedded in paraffin, or fixed in OCT. Stepwise sections were cut and stained with H&E. Colonic inflammation, dysplasia and neoplasms were graded based on described criteria (Suzuki et al. (2004) Cancer Sci, 95, 721-7) by a pathologist blinded to the conditions. Immunochemical analysis was done as described below.

### Immunochemical Analysis

Immunochemical analysis of frozen human tissue sections was done as described earlier (Srikrishna et al. (2001) Journal of Immunology, 166, 624-632) except for the following modifications: Before fixing in formalin, sections were immersed in 0.03% H2O₂ for 30 minutes at room temperature, blocked with 1% BSA/PBS for 20 minutes followed by 0.1% avidin and 0.01% biotin in succession for 15 minutes at room temperature in a humid chamber with PBS washes in between. The following antibodies were used for staining: mAbGB3.1, anti-human RAGE (11F2, kind gift of NOVARTIS® Foundation, Japan) or commercially available anti-human S100A9. After counterstaining, the slides were scanned into APERIO® Imaging system and were also observed under a brightfield microscope and images acquired as given below.

Fixed mouse colon sections (6 μm) were deparafinized and rehydrated in PBS; endogenous peroxidases were neutralized with 1% hydrogen peroxide and blocked with commercially available avidin/biotin. Samples were then incubated with commercially available anti-mouse S100A8 or anti-mouse S100A9 (goat polyclonal), followed by biotin-conjugated secondary antibody. Binding was detected using commercially available streptavidin-peroxidase complex and diaminobenzidine (DAKO®). Sections were then counterstained with hematoxylin. To characterize leukocyte populations, sections were stained with anti-mouse CD11b, anti-mouse Gr-1 (commercially available) or both followed by ALEXA FLUOR® 594 or ALEXA FLUOR® 488 conjugated secondary antibodies (commercially available), and cover slipped with commercially available mounting medium. Slides were examined using an Inverted TE300 NikonWide Field and Fluorescence Microscope and images were acquired with a CCD SPOT RT Camera (Diagnostic Instruments Inc.) using SPOT advanced software.

### Cells

HT-29, Caco-2 and CT-26 tumor cell lines were obtained from American Type Culture Collection (ATCC) and maintained in Dulbecco's modified Eagle's medium containing glutamine, penicillin and streptomycin and 10% FBS. Cells were harvested using PBS with 5mM EDTA. Cell membranes were generated by homogenization in PBS with commercially available protease inhibitors. Nuclei and cell debris were removed by centrifugation at 300×g for 10 min at 4° C. Resulting supernatants were ultracentrifuged at 110,000×g for 30 min at 4° C., pellets resuspended in 200 μl of PBS, protease inhibitors and 1% NP-40 and membrane proteins were extracted by slow stirring overnight at 4° C. Proteins were subjected to deglycosylation using PNGaseF. For mAbGB3.1 immunoprecipitation, membrane proteins were incubated with mAbGB3.1 coupled Affigel beads in PBS. After overnight incubation at 4° C., the individual pellets were washed to remove unbound label, and bound proteins were eluted with 0.2% SDS/1% 2-ME.

Purification of Bovine RAGE and mAbGB3.1 Enrichment

Fresh frozen bovine lung was homogenized in 20 mM Tris HCl, pH 7.4 containing containing protease inhibitors, 10 mM DTT, 1 mM CaCl₂, and 1% Nonidet P-40 (NP-40). The suspension was centrifuged at 650×g for 15 min and then at 10,000×g for 30 min and the supernatant was enriched for RAGE using sequential ammonium sulfate precipitation (33% followed by 50%). Precipitate obtained from 50% ammonium sulfate was collected by centrifugation at 10,000×g for 30 min, dissolved in PBS with 1% NP-40 and dialyzed extensively against the same buffer to remove ammonium sulfate. RAGE was further purified by first pre-clearing over rat IgG immobilized protein G Sepharose followed by affinity purification over rat monoclonal anti-RAGE (11F2) immobilized Protein G Sepharose. Bound RAGE was eluted with 0.1M triethanolamine, pH 11.5 and neutralized using IM Tris-HCl, pH 7.5. Contaminant bovine IgG was removed by passing through Protein G Sepharose. Homogeneity was assessed on SDS PAGE gels as described below. For mAbGB3.1 enrichment, the purified protein was incubated with mAbGB3.1 immobilized Affigel-10 beads twice, and bound protein was released using base as above.

### Electrophoresis and Western Blots

20 μg of membrane proteins from tumor cells before and after deglycosylation or membrane proteins immunoprecipitated by mAbGB3.1 from 1 mg starting material were electrophoresed on denaturing and reducing 12% polyacrylamide gels, and transferred to nitrocellulose membranes. The blots were blocked with 10% dry skimmed milk, washed and incubated with a rabbit polyclonal anti-RAGE followed by alkaline phosphatase-conjugated secondary antibody. Staining was visualized using commercially available BCIP®/NBT. 1 μg purified RAGE or 5 μg purified S100A8/A9 were analyzed on 12% or 17% SDS PAGE gels respectively and stained by Coomassie Brilliant Blue. Purified RAGE was examined by western blot using anti-RAGE before and after EndoH and PNGaseF deglycosylation and before and after mAbGB3.1 immunoprecipitation.

### Ligand Binding Assay

S100A8/A9 proteins were purified as described earlier (Hunter et al. (1998) J Biol Chem, 273, 12427-35) and purity assessed by gels. The complex was added at increasing concentrations to the wells of a 96-well ELISA plate containing immobilized total bovine RAGE, RAGE deglycosylated under non-denaturing conditions using PNGaseF or mAbGB3.1 enriched RAGE. Incubations were done in HBSS containing 1 mM CaCl₂ and 1% BSA overnight at 4° C. Bound S100A8/A9 was quantified using goat anti-S100A8, followed by anti-rabbit IgG alkaline phosphatase conjugate and p-nitrophenyl phosphate substrate, against standard S100A8/A9. RAGE bound to the plates was quantified independently using anti-RAGE. Non-specific binding was determined by incubation of S100A8/A9 in wells blocked with BSA alone or in wells coated with mock immunoprecipitates of mAbGB3.1 and blocked with BSA. Non-linear regression analysis was done using GRAPHPAD PRISM®.

For radio-labeled protein binding assay, purified mouse S100A8/A9 heterodimeric complex was labeled with Na¹²⁵I using commercially available lodobeads, as per the manufacturer's protocol. CT-26 colon carcinoma cells were harvested using PBS containing 10 mM EDTA, and were stripped of surface bound endogenous S100A8/A9 by brief incubation in 50 mM glycine, 100 mM NaCl, pH 3.0 for 3 min at 4° C. followed by neutralization with cold HBSS. Cells were incubated with increasing concentrations of radio-labeled S100A8/A9 for 1 h at 4° C. in HBSS. They were then washed twice with 1 ml of HBSS, solubilized in 0.2 ml of 0.5 M NaOH and cell-bound radioactivity was counted. Non-specific binding was determined by binding in the presence of 50-fold molar excess of cold ligand. Where indicated, binding was carried out in the presence of 10-fold molar excess of mAbGB3.1, isotype control antibody, anti-RAGE or anti-S100A8. Saturation binding kinetic analyses were performed using GRAPHPAD PRISM®. Values were normalized for number of cells.

### NF-κB Reporter Assay

CT-26 cells were cultured overnight in 24-well plates (2×10⁵ cells/well). Cells were transiently transfected with 1 μg of plasmid DNA comprising 0.1 μg of PNF-κB-Luc, containing a Luciferase cDNA under a regular TATA Box and an enhancer element with five NF-κB binding sites, (Stratagene), 0.1 μg of pRL-TK construct (containing Renilla reniformis luciferase gene under the thymidine kinase promoter (Promega), and inert filler plasmid, using Lipofectamine 2000 (Invitrogen) according to the manufacturer's instructions. 6 h after transfection, cells were placed in low serum medium for 18 h, and stimulated with endotoxin-free S100A8/A9 (0.5 ug/ml, ˜20 nM) in the presence or absence of inhibitors. 24 h after activation, cells were harvested and enzymes were measured in lysates. The luciferase activities were determined using the DUAL-LUCIFERASE® reporter Assay System (PROMEGA®) according to the manufacturer's protocol. Transfected unactivated cells accounted for endogenous activity.

### Cell Proliferation Assay

CT-26 cells were plated in 96-well culture plates in 100 μl of 1% serum medium and grown with and without mouse S100A8/A9 in the presence or absence of mAbGB3.1, anti-RAGE or control antibody. Proliferation was measured using MTS assay (CELLTITER96® Aqueous One cell proliferation, PROMEGA®) as per manufacturer's instructions.

### Serum Cytokines

Serum TNFα and IL-6 were measured using commercially available ELISA kits as per manufacturer's protocol.

### Statistical Analysis

Statistical comparisons were performed using one-way analysis of variance (ANOVA) or Student's t test. Differences were considered statistically significant when p <0.05.

Examples 8-14 described below demonstrate the pro-inflammatory S100 proteins regulate the accumulation of myeloid-derived suppressor cells.

### EXAMPLE 8

### MDSC in the Blood, Spleen, and Bone Marrow of 4T1 Tumor-Bearing Mice, and Metastatic 4T1 Tumors have a Similar Phenotype and Suppressive Activity

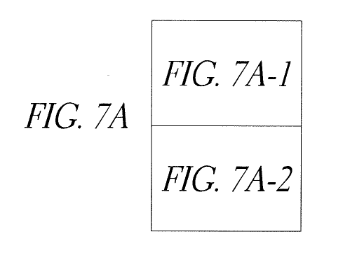

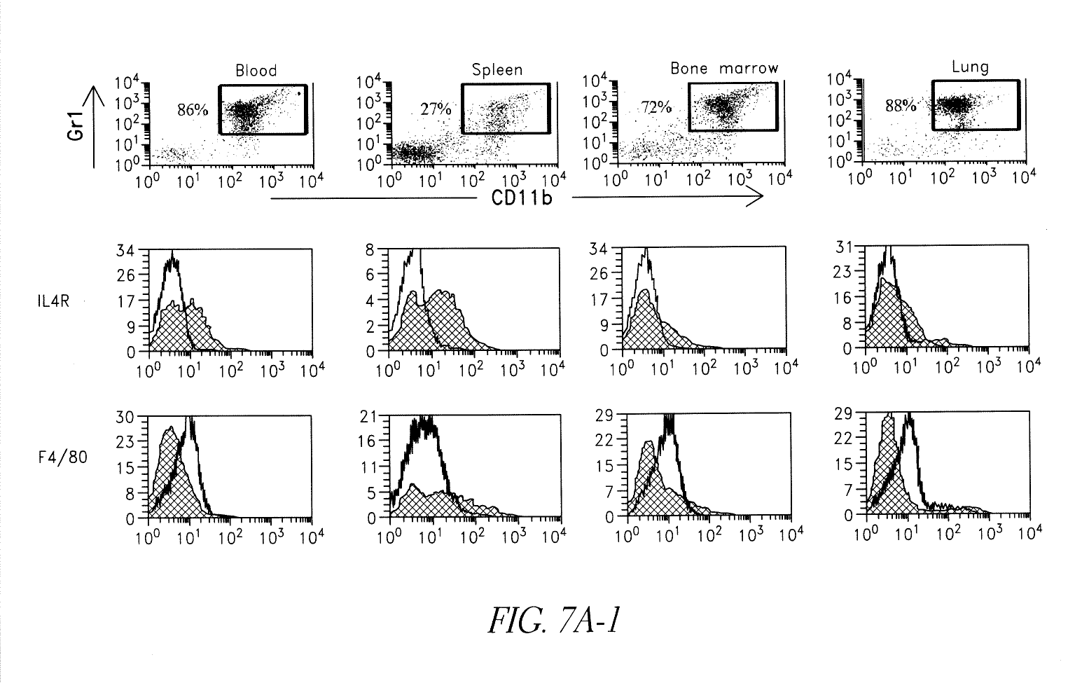

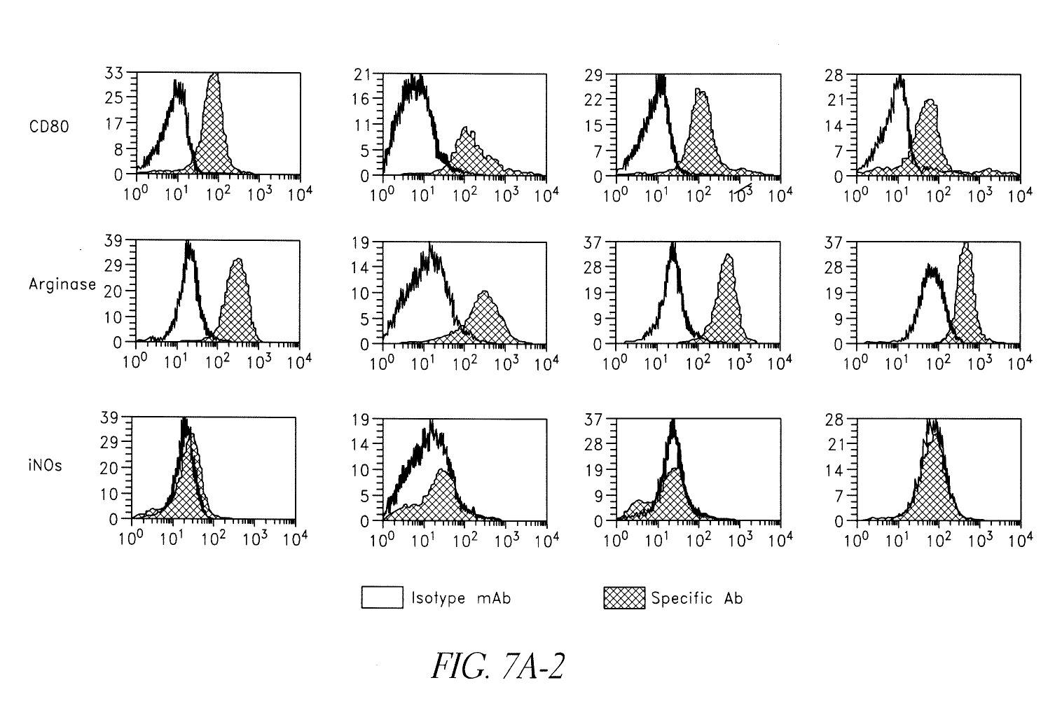

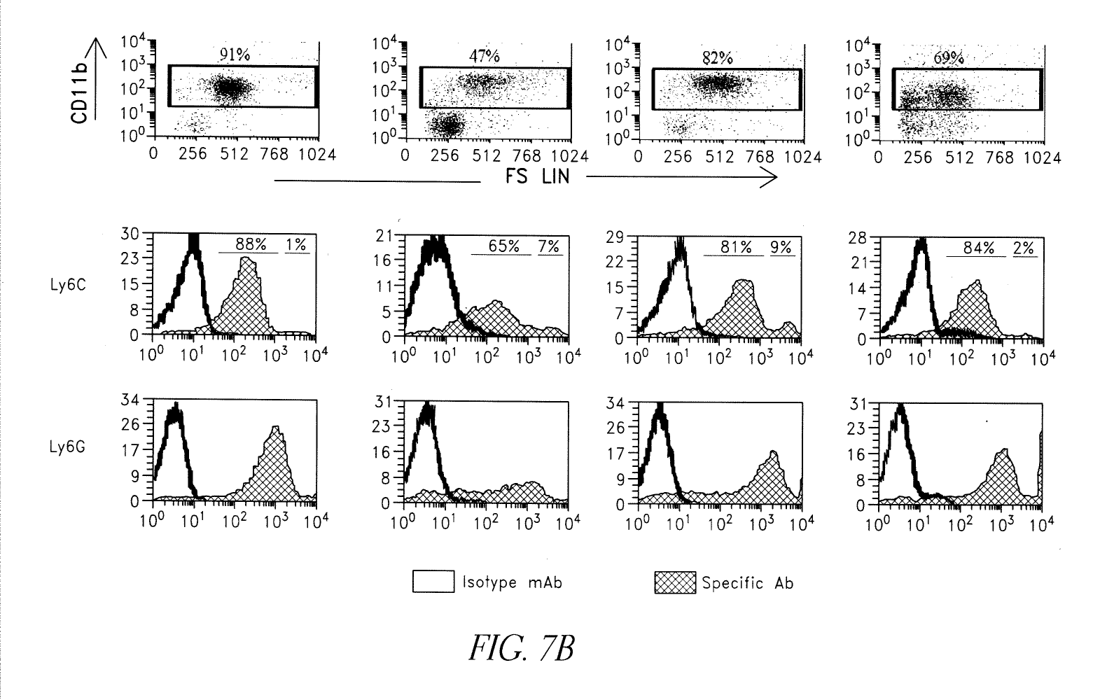

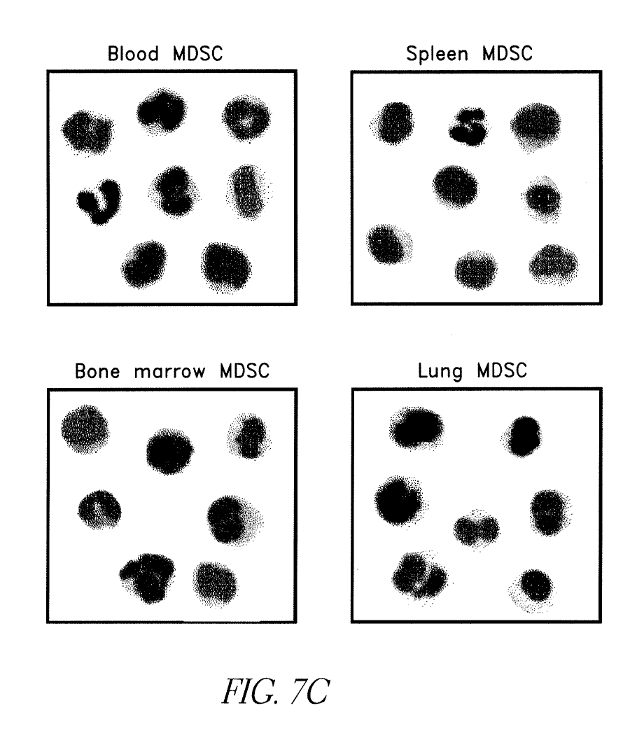

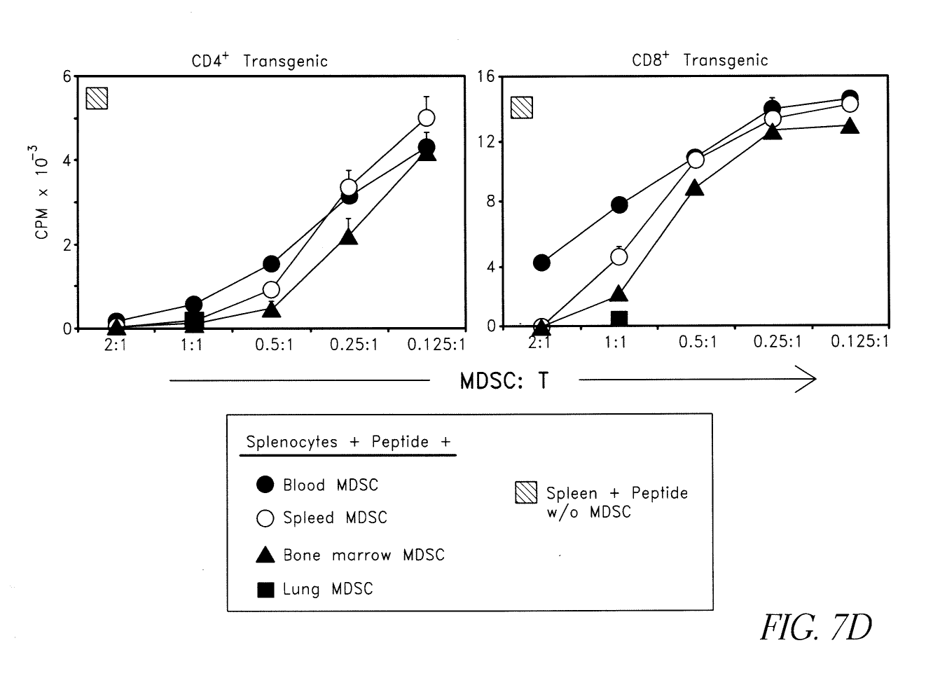

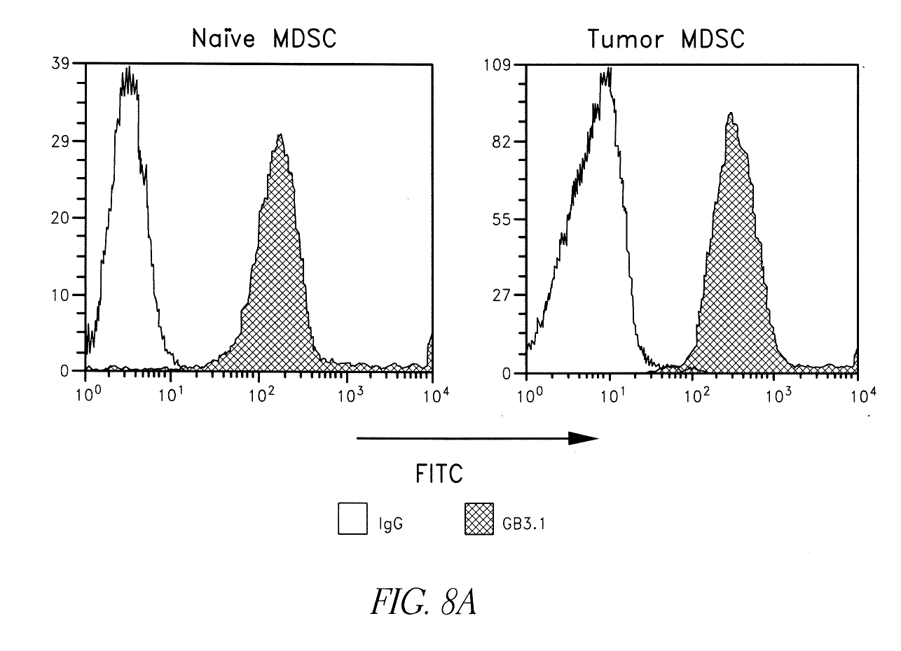

MDSC accumulate at the site of tumor and are elevated in the blood, bone marrow, and spleens of tumor-bearing mice. To confirm that MDSC within a given individual are homogeneous regardless of location, splenocytes, circulating leukocytes (blood), bone marrow cells, and lungs containing metastases were isolated from BALB/c mice that had been inoculated in the mammary fat pad 33 days earlier with 7000 4T1 tumor cells. The resulting cells were analyzed by immunofluorescence and flow cytometry for the canonical MDSC markers Gr1 and CD11b, and gated Gr1⁺CD11b⁺ cells were further analyzed for F4/80, IL-4Rα, and CD80, markers that are differentially expressed on MDSC isolated from mice with histologically distinct tumors (Umemura et al. (2008) J Leukoc Biol 83:1136; Serafini et al. (2006) Semin Cancer Biol 16:53). The percentage of Gr1⁺CD11b⁺ cells differed in each tissue; however, Gr1⁺CD11b⁺ cells from all locations shared a similar phenotype (Gr1^{hi}CD11b^{hi}F4/80⁻CD80⁺IL-4Ra^{+/-}Arginase⁺) (FIG. 7A). Previous studies have classified Gr1⁺CD11b⁺ cells as either “neutrophil-like” or “monocyte-like” based on the expression of Ly6G (immature neutrophil marker) or Ly6C (monocyte/macrophage marker), respectively, and nuclear morphology (Umemura et al. (2008) J Leukoc Biol 83:1136; Rossner et al. (2005) Eur J Immunol 35:3533; Movahedi et al. (2008) Blood 111:4233; Sawanobori et al. (2008) Blood). To determine if differentially localized Gr1⁺CD11b⁺ cells are distinguished by either of these categories, CD11b⁺ cells were gated and analyzed for Ly6G and Ly6C (FIG. 7B), and Gr1⁺CD11b⁺ cells were Wright-Giemsa stained and examined histologically (FIG. 7C). All MDSC express Ly6C and Ly6G, although MDSC from bone marrow and spleen of 4T1-tumor-bearing mice have small subpopulations (9% and 7%, respectively) of Ly6C^{hi} cells. As previously reported for blood MDSC (Sinha et al. (2007) J Immunol 179:977), 4T1-induced Gr1⁺CD11b⁺ cells from all locations are a mixture of cells with single and multi-lobed nuclei (FIG. 7B) To confirm that MDSC are consistently suppressive regardless of their location, MDSC from blood, spleen, bone marrow, and lung (all ≧85% Gr1⁺CD11b⁺) were co-cultured at varying ratios with splenocytes from transgenic D011.10 (CD4⁺) or Clone 4 (CD8⁺) mice and their respective peptides, and T cell proliferation was measured by uptake of 3H-thymidine (FIG. 7D). These results, combined with previous studies demonstrating that splenic MDSC from tumor-free mice are equally suppressive on a per cell basis as splenic MDSC from tumor-bearing mice (Sinha et al. (2005) J Immunol 174:636), demonstrate that MDSC from different anatomical locations have approximately equivalent suppressive activity. Therefore, anatomical location does not affect the phenotype or function of MDSC, and subsequent experiments performed with blood MDSC are representative of MDSC from all locations.

### EXAMPLE 9

### MDSC have Receptors for the S100A8/A9 Pro-Inflammatory Proteins

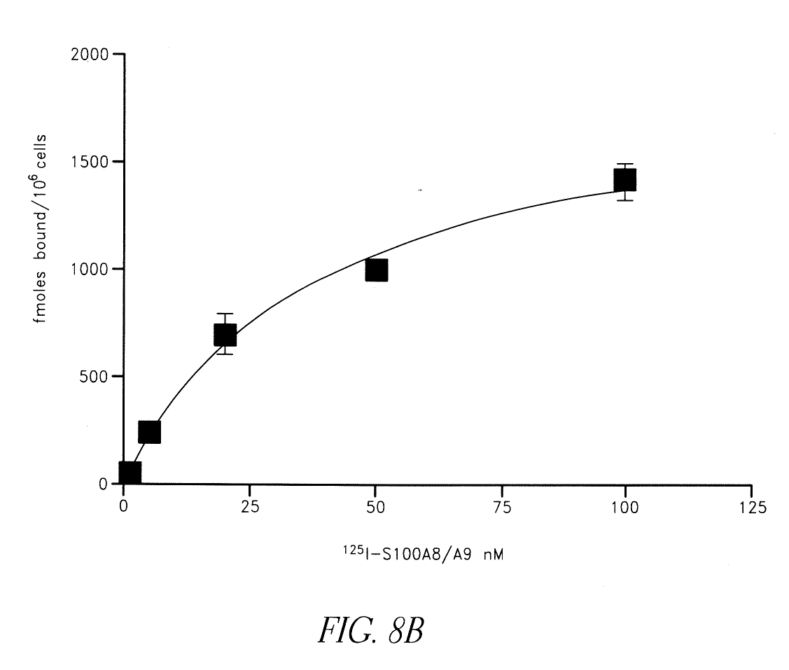

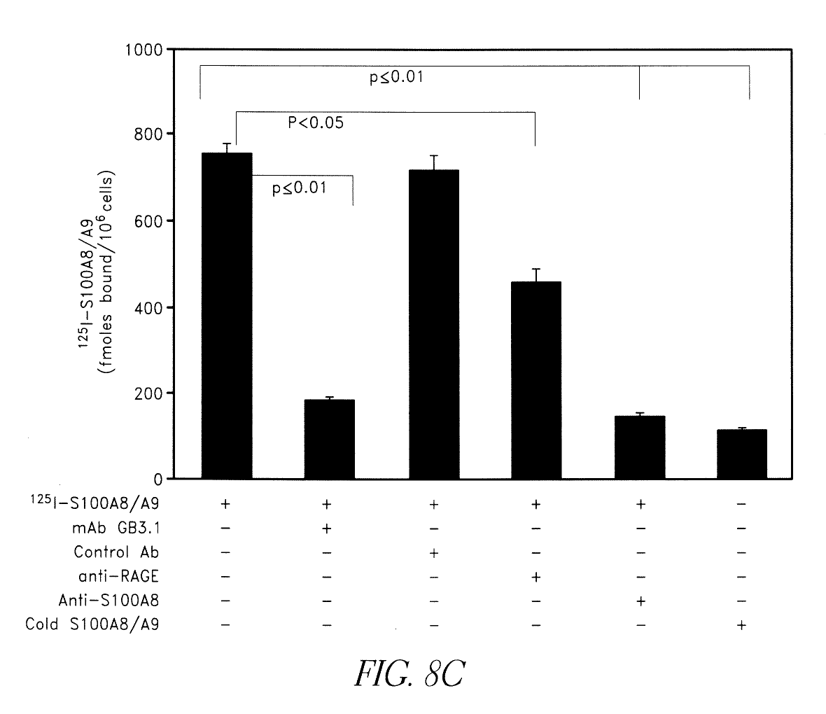

Upon cellular activation, S100A8 and S100A9 proteins are secreted into the extracellular milieu where they mediate leukocyte recruitment and other functions that promote inflammation (Roth et al. (2003) Trends Immunol 24:155). Since inflammation is an inducer of MDSC (Bunt et al. (2006) J Immunol 176:284; Sinha et al. (2007) Cancer Res 67:4507; Song et al. (2005) J Immunol 175:8200) and pro-inflammatory S100 proteins are present in the tumor environment of mammary carcinomas (Seth et al. (2003) Anticancer Res 23:2043; Carlsson et al. (2005) Int J Oncol 27:1473), it was examined if MDSC express receptors for S100 proteins. Blood was collected from tumor-free mice and from BALB/c mice 31 days after 4T1 inoculation, when metastatic disease was established and primary tumors were 9.41±0.3 mm in diameter. Gr1⁺CD11b⁺ leukocytes from both tumor-free and tumor-bearing mice stained with mAbGB3.1 which detects carboxylated N-glycans that are part of the cellular receptor for S100A8/A9 proteins (Srikrishna et al. (2001) J Immunol 166:4678; Srikrishna et al. (2001) J Immunol 166:624) (FIG. 8A). Cell membranes prepared from blood MDSC (>90% Gr1⁺CD11b⁺), incubated with increasing concentrations of radio-iodinated S100A8/A9 proteins had specific saturable binding sites with a Kd of 37.43±6.99 nM and a Bmax of 1.882±0.142 pmoles/million cells (FIG. 8B). ¹²⁵I-labeled S100A8/A9 binding was displaced by molar excess of cold ligand or by anti-S100A8 antibodies (FIG. 8C). To ascertain that the S100A8/A9 heterodimers were binding to carboxylated N-glycans on cell surface receptors, MDSC membranes were incubated with ¹²⁵I-labeled S100A8/A9 proteins in the presence of mAbGB3.1. Binding was significantly reduced in the presence of mAbGB3.1 (FIG. 8B), while an isotype control antibody had no effect on binding. Interestingly, anti-RAGE also partially blocked binding, consistent with reports that RAGE is modified by carboxylated N-glycans (Srikrishna et al. (2002) J Neurochem 80:998), and that S100A8/A9 proteins bind to RAGE as described herein. Therefore, MDSC from tumor-free mice and from mice with primary and metastatic mammary carcinoma contained carboxylated N-glycan receptors that bound S100A8/A9, consistent with the concept that pro-inflammatory S100 proteins are part of the inflammatory milieu that induces the accumulation of MDSC.

### EXAMPLE 10

### MDSC Synthesize and Secrete S100A8/A9 Heterodimers

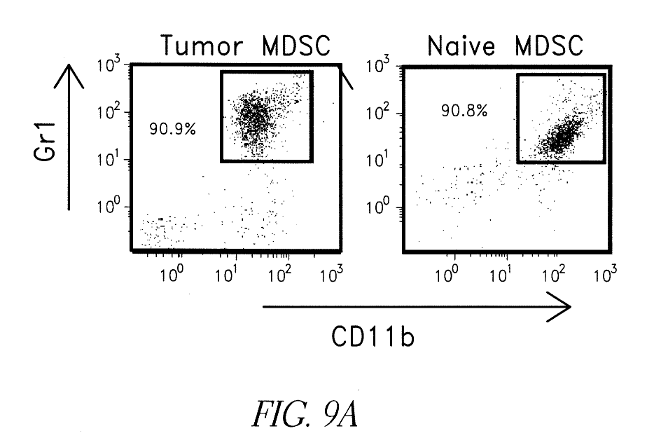

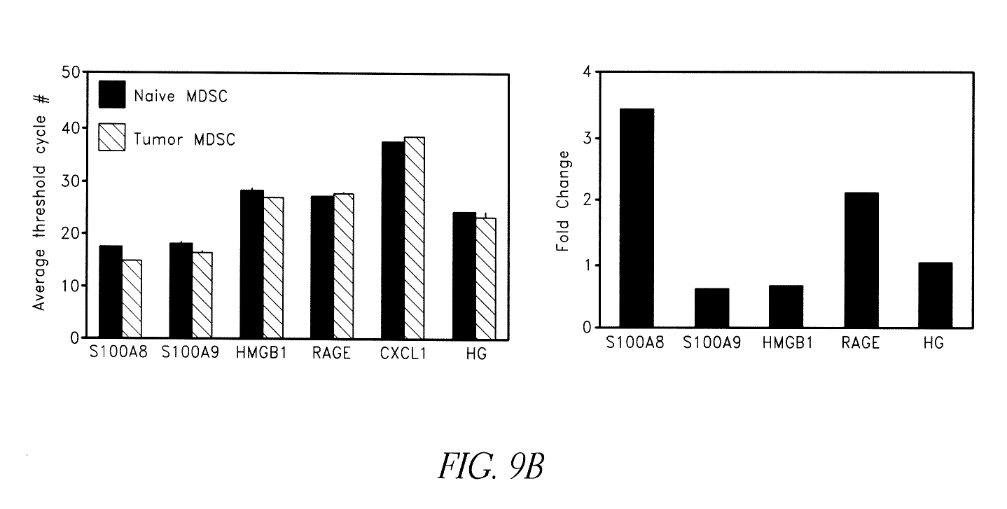

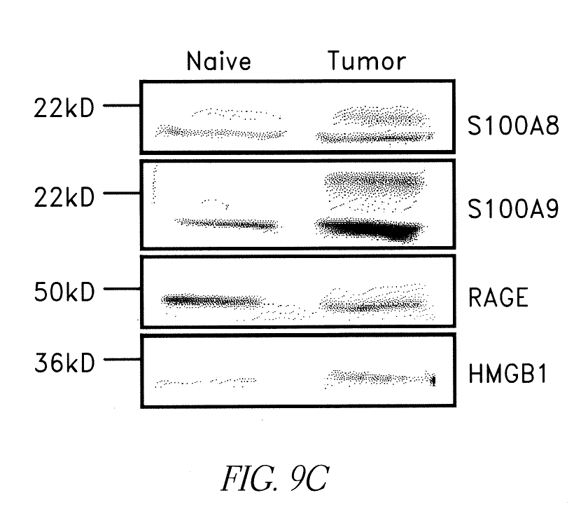

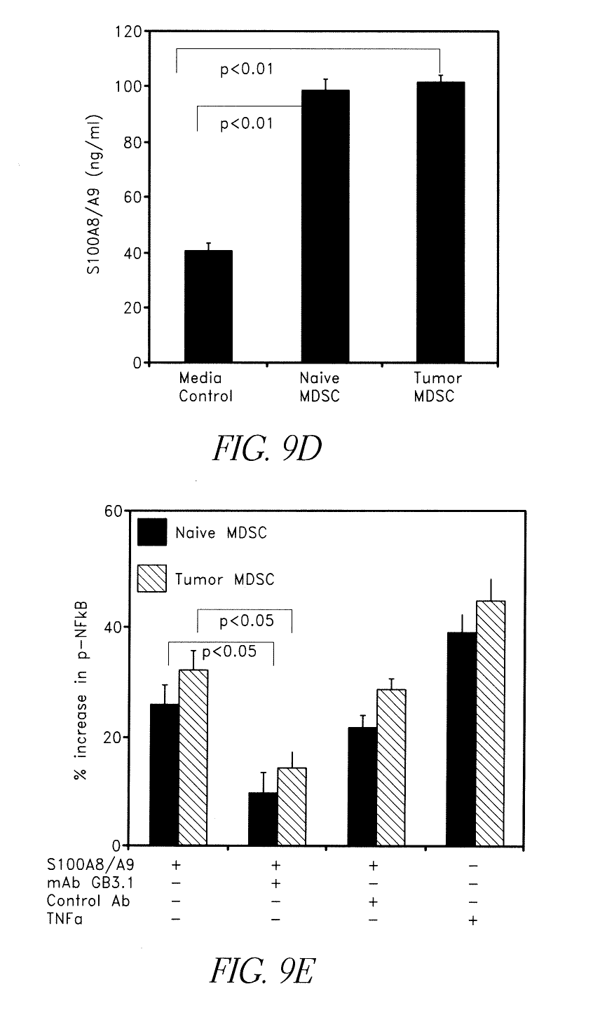

Since S100A8/A9 proteins are synthesized by cells of the myeloid lineage, the present studies sought to answer whether MDSC may also contribute to the inflammatory milieu by producing these pro-inflammatory mediators. To test this hypothesis, mice were bled on day 36 after 4T1 inoculation when Gr1⁺CD11b⁺ cells were >90% of the circulating white cells (FIG. 9A, left panel), primary tumors were 9.70±0.21 mm in diameter, and metastatic disease was established. Tumor-free mice were also bled and Gr1⁺CD11b⁺ cells, comprising <10% of the circulating white cells, were purified to >90% purity (FIG. 9A right panel). To determine if the Gr1⁺CD11b⁺ cells express S100A8/A9, real time RT-PCR was performed and the number of cycles required to amplify these messages was compared to that required for a panel of five housekeeping genes and the poorly expressed CXCL1 gene (FIG. 9B left panel). Gr1⁺CD11b⁺ cells from both tumor-free and tumor-bearing mice expressed elevated levels of S100A8/A9 relative to the housekeeping genes (FIG. 9B). RAGE and HMGB1 transcripts were also expressed; however, at somewhat lower levels than the housekeeping genes. S100A8 and RAGE were increased 2-3 fold in Gr1⁺CD11b⁺ cells from tumor-bearing mice vs. tumor-free mice (FIG. 9B, right panel).

To confirm the PCR results, lysates of the purified MDSC were analyzed by western blotting for S100A8/A9, HMGB1, and RAGE (FIG. 9C). S100A8 and S100A9 monomers and low levels of HMGB1 were present in Gr1⁺CD11b⁺ cells from both tumor-bearing and tumor-free mice. HMGB1 levels in MDSC increase in the presence of tumor, and multimers of S100A8/A9, which are the active forms, were only present in MDSC from tumor-bearing mice. Therefore, MDSC from tumor-bearing mice, but not from tumor-free mice contain functionally active S100A8/A9 proteins, consistent with the concept that these pro-inflammatory mediators and receptors of MDSC are regulated by tumor.

To test whether MDSC secrete S100A8/A9 proteins, circulating white blood cells from BALB/c mice with advanced 4T1 tumors (>95% Gr1⁺CD11b⁺ cells) and purified MDSC from tumor-free mice (>95% Gr1⁺CD11b⁺) were cultured for 24 hrs and the supernatants assayed by ELISA for S100A8/A9 proteins. MDSC from both tumor-bearing and tumor-free mice released significant amounts of S100A8/A9 proteins (FIG. 9D); however, the released S100 proteins from tumor-free mice are unlikely to be functional since MDSC from tumor-free mice do not contain multimers (see FIG. 9C). Therefore, MDSC from tumor-bearing mice not only have the capacity to respond to S100A8/A9 proteins, but they may also sustain an autocrine stimulatory pathway by secreting active S100A8/A9 proteins.

### EXAMPLE 11

### S100A8/A9 Signal Through the NF-κB Pathway in MDSC

To determine if S100A8/A9 proteins activate MDSC after binding to cell surface receptors, potential signaling pathways in the Gr1⁺CD11b⁺ cells were examined. Circulating white blood cells were obtained from BALB/c mice on day 31 after 4T1 inoculation (>95% Gr1⁺CD11b⁺ cells) and were purified from tumor-free mice (>95% Gr1⁺CD11b⁺), co-cultured with endotoxin-free S100A8/A9 proteins, and cell lysates assayed by ELISA for phosphorylated NF-κB and MAPKinase pathway signaling molecules, two pathways activated by inflammation. None of the molecules in the MAPKinase pathway were affected. However, NF-κB was phosphorylated in MDSC from both tumor-bearing and tumor-free mice within the first 10 minutes of incubation at a level comparable to activation by TNF-α, a NF-κB-dependent gene that is known to be activated by S100A8/A9 (Srikrishna et al. (2005) J Immunol 175:5412) (FIG. 9E). S100A8/A9-induced activation was reduced in the presence of mAbGB3.1. Therefore, S100A8/A9 proteins activate the NF-κB pathway in MDSC, and this activation is mediated in part by binding to cell surface receptors expressing carboxylated glycans.

### EXAMPLE 12

### Blocking of S100A8/A9 Binding Reduces the Levels of MDSC and S100A8/A9 Proteins in the Blood of Tumor-Bearing Mice

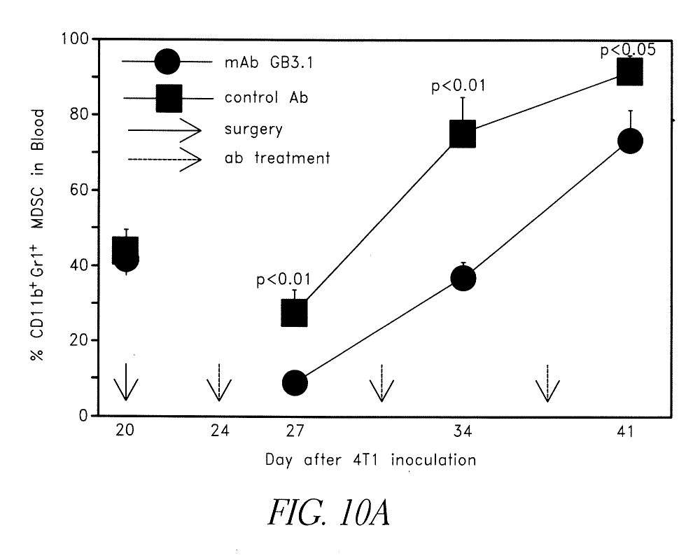

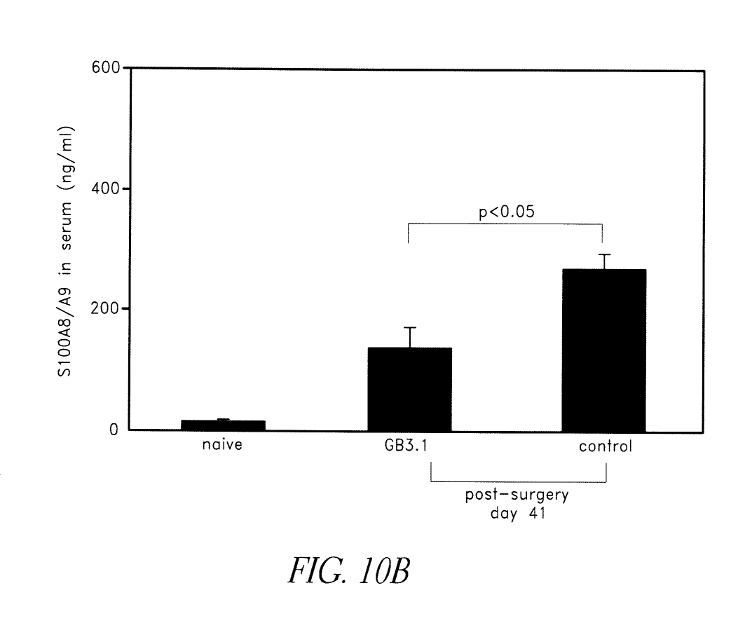

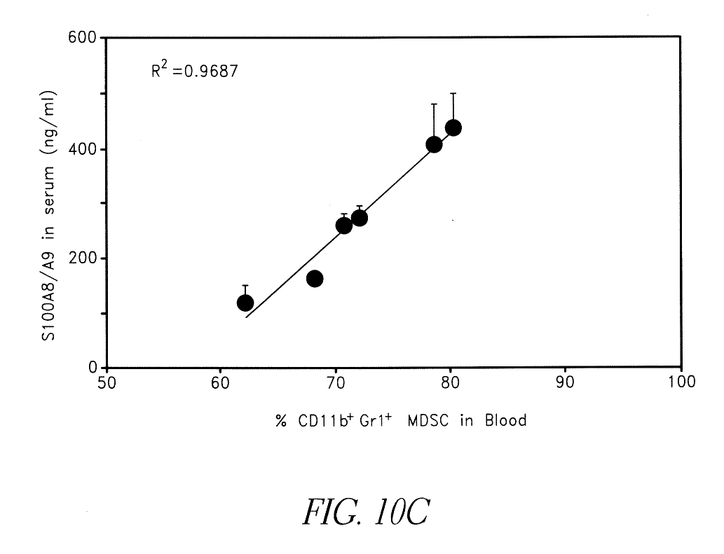

If S100A8/A9-mediated signaling promotes recruitment of MDSC, then inhibition of S100A8/A9 binding in vivo may reduce MDSC levels. To test this possibility mAbGB3.1 or irrelevant control mAb was administered to BALB/c mice with established metastatic disease whose primary 4T1 tumors were surgically removed. This experimental design was used for two reasons: (i) It was previously demonstrated that MDSC levels decrease with removal of primary tumor, and rapidly increase thereafter unless there is therapeutic intervention (Sinha et al. (2007) J Immunol 179:977; Sinha et al. (2005) Cancer Immunol Immunother 54:1137). (ii) This setting closely models the conditions under which immunotherapy would be administered clinically and the present studies determined if mAbGB3.1 could facilitate a reduction in MDSC in a clinically relevant situation. Mice were inoculated on day 0 with 4T1 tumor cells, primary tumors were surgically resected on day 20, and mAbGB3.1 or irrelevant isotype control antibody treatment was started on day 24. Primary tumor diameters (5.09±0.76 and 5.36±0.68 mm) and percent MDSC in blood (41.25%±3.98 and 44.65%±6.60) were matched at the time of surgery for mAbGB3.1 and control antibody treated groups, respectively. Mice were bled 3 days after each antibody treatment, and their circulating white blood cells were stained for Gr1 and CD11 b. Consistent with previous observations, removal of primary tumor temporarily reduced MDSC levels in the blood (Sinha et al. (2005) J Immunol 174:636). Treatment with mAbGB3.1 significantly decreased the accumulation of MDSC relative to treatment with control mAb (FIG. 10A), and did not affect the levels of other peripheral blood monocytes, dendritic cells, T cells or B cells.

If MDSC production of S100A8/A9 proteins contributes to the overall inflammatory milieu in tumor-bearing individuals, then these proteins should be elevated in tumor-bearing mice and reduction of MDSC should result in a concomitant decrease in serum S100A8/A9. To test this hypothesis, the levels of S100A8/A9 proteins were measured in the serum of tumor-free and tumor-bearing mice treated with mAbGB3.1 or control mAb. Treatment with mAb GB3.1 significantly reduced serum levels of S100A8/A9 in tumor-bearing mice (FIG. 10B), and the level of serum S100A8/A9 in tumor-bearing mice was roughly proportional to the quantity of MDSC in the blood (FIG. 10C). mAbGB3.1-treatment did not affect the amount of S100A8/A9 released per cell in culture. Therefore, the accumulation of S100A8/A9 in the serum of tumor-bearing mice may be at least partially due to the production of these molecules by MDSC.

### EXAMPLE 13

### S100A8/A9 Proteins are Chemotactic for MDSC

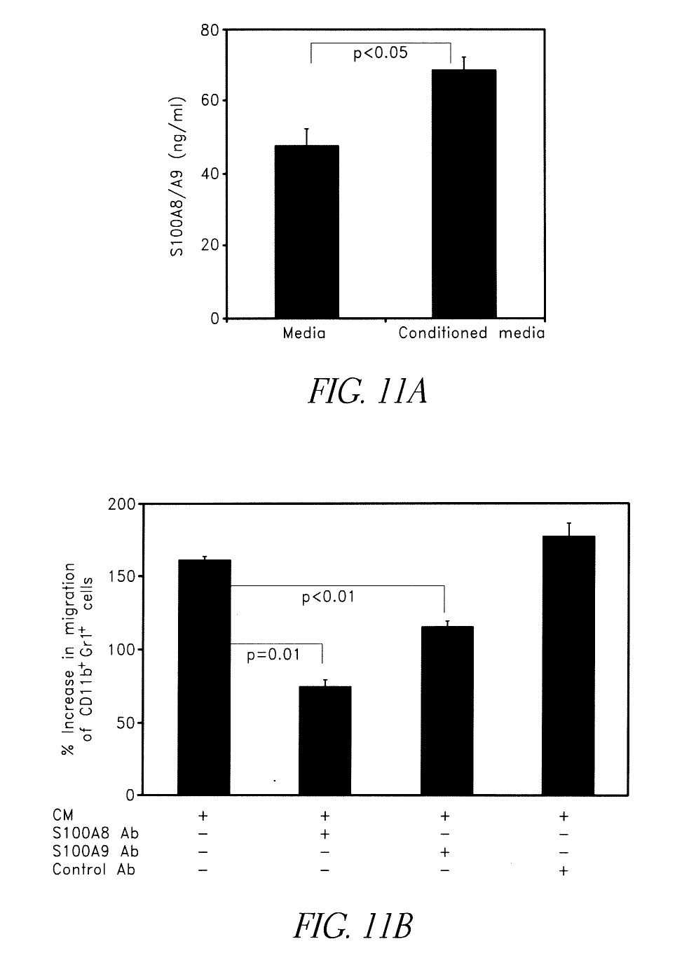

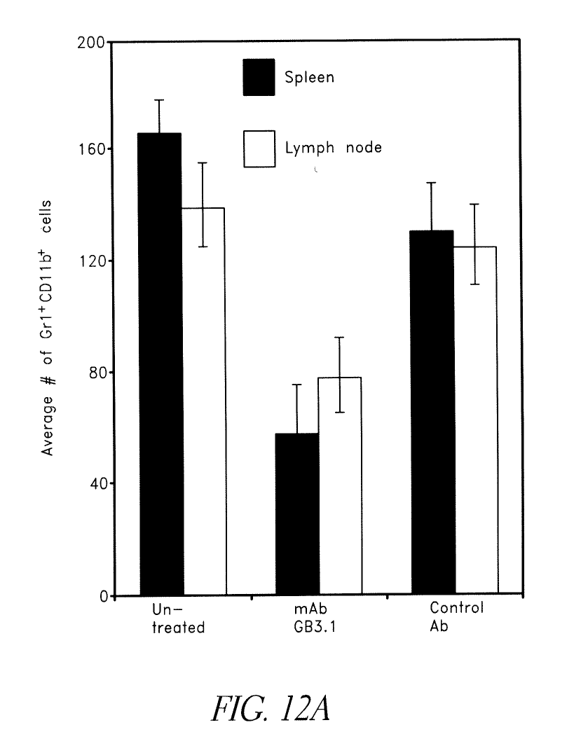

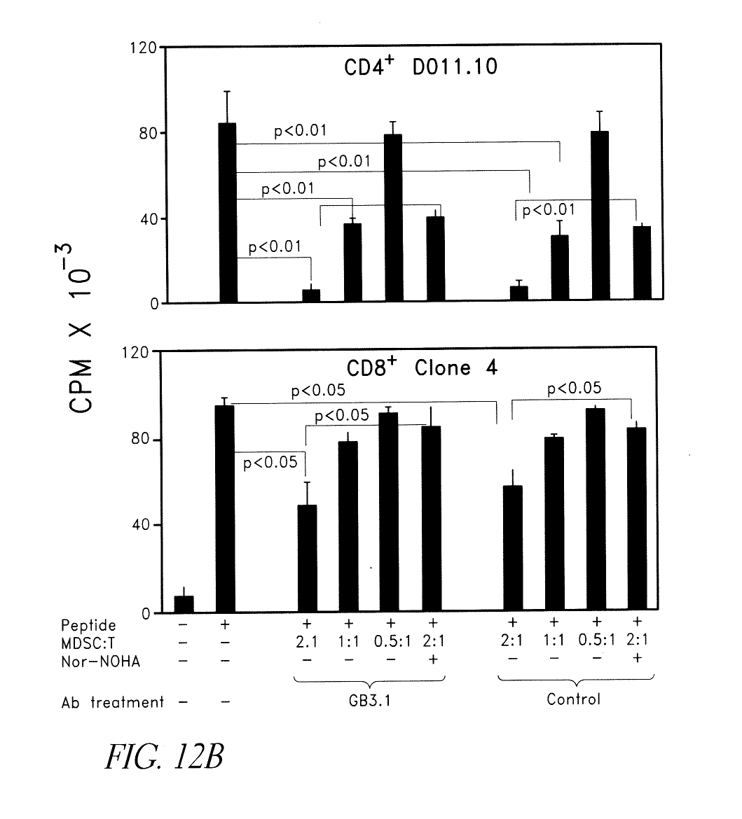

To test whether tumor cell-secreted S100A8/A9 proteins attract MDSC to tumor sites, supernatants from 4T1 cultures were assayed for S100A8/A9 and MDSC were tested for migration in response to 4T1 supernatants. 4T1 cells produce S100A8/A9 proteins (FIG. 11A) and 4T1 supernatants are chemoattractants for MDSC (FIG. 11B). S100A8/A9 contributed to the chemotaxis mediated by 4T1 supernatants because the addition of antibodies to S100A8/A9 significant decreased MDSC migration.

### EXAMPLE 14

### In Vivo Treatment with mAbGB3.1 Reduces the Accumulation of MDSC

In addition to accumulating in the blood, spleen, bone marrow, and tumor sites, MDSC are also found in the lymph nodes of tumor-bearing mice (Sica et al. (2007). J Clin Invest 117:1155). To determine if blockade of S100A8/A9 receptors reduces MDSC levels in sites other than the blood, BALB/c mice were inoculated with 4T1, primary tumor was surgically resected on day 20, and mAbGB3.1 or control antibody treatment was started on day 24. Secondary lymphoid organs and metastatic lungs were cryo-preserved on day 41, when mice had established primary tumors and metastatic disease, and were stained for Gr1⁺CD11b⁺ cells. Gr1⁺CD11b⁺ cells were present in high numbers in the lymph nodes and spleens of untreated mice, and mAbGB3.1 treatment reduced accumulation of these cells at these sites (FIG. 15A), but not in the lungs.

In addition to increasing the numbers of MDSC, S100A8/A9 proteins may increase MDSC suppressive activity. In this case, MDSC of tumor-bearing mice treated with mAbGB3.1 might be less suppressive on a per cell basis than MDSC of control antibody-treated mice. To test this hypothesis, mice were inoculated on day 0 with 4T1 cells, primary tumors were removed on day 26, mAbGB3.1 or control antibody treatment was started on day 29, and splenic MDSC were harvested, purified (>90% Gr1⁺CD11b⁺), and tested for their ability to suppress peptide-activation of CD4⁺ DO1.10 or CD8⁺ Clone 4 transgenic T cells (FIG. 15B). T cell activation was measured by ³H-thymidine incorporation. Gr1⁺CD11b⁺ MDSC from mAbGB3.1-treated and from control-treated mice were equally suppressive on a per cell basis, and both MDSC cell populations used arginase to mediate their suppressive effects, as shown by restoration of T cell proliferation in the presence of the arginase inhibitor, nor-NOHA. Therefore, S100A8/A9 proteins promote immune suppression by enhancing the recruitment and accumulation of Gr1⁺CD11b⁺ cells, but do not alter the suppressive activity of individual MDSC.

Thus, Examples 8-14 report the discovery that S100A8/A9 pro-inflammatory mediators not only induced the accumulation of MDSC, but they were also secreted by MDSC and bound to cell surface receptors leading to signaling within MDSC. Thus, the S100A8/A9 proteins provide for a positive autocrine feedback loop that ensures the maintenance of functionally suppressive MDSC within an inflammatory tumor environment.

As reported earlier, Gr1⁺CD11b⁺ MDSC from tumor-free mice and from mice with 4T1 tumors are equally suppressive on a per cell basis (Sinha et al. (2005) J Immunol 174:636). This observation combined with the current findings that MDSC from both sources share a common phenotype and are both activated by S100A8/A9 through NF-κB, supports the discovery that tumors regulate the expansion of a normal myeloid cell population rather than induce a novel myeloid population. Indeed, multiple pro-inflammatory mediators including S100A8/A9 (as shown herein), IL-1β (Bunt et al. (2006) J Immunol 176:284; Song et al. (2005) J Immunol 175:8200), IL-6 (Bunt et al. (2007) Cancer Res 67:10019), and prostaglandin E2 (Sinha et al. (2007) Cancer Res 67:4507; Rodriguez et al. (2005) J Exp Med 202:931) up-regulate MDSC, indicating that inflammation is an important contributing factor to the accumulation of MDSC in tumor-bearing individuals.

The section below describes in greater detail some of the materials and methods used in Example 8-14. Mice

BALB/c and transgenic DO11.10 (I-A^{d}-restricted, ovalbumin (OVA) peptide 323-339-specific; (Sinha et al. (2005) J Immunol 174:636)) and clone 4 (H-2K^{d}-restricted, hemagglutinin (HA) peptide 518-526-specific; (Sinha et al. (2005) J Immunol 174:636)) mice were bred in the University of Maryland Baltimore County animal facility. All animal procedures were approved by the UMBC Institutional Animal Care and Use Committee.

### Antibodies and Flow Cytometry

Fluorescently-coupled Gr1, CD11b, IL-4Rα, CD80, arginase, iNOs, Ly-6G, Ly-6C and isotype control antibodies were commercially available). F4/80 and Fc block antibody were commercially available. S100A8 and S100A9 antibodies were commercially available. Commercial antibodies were used at a concentration of 4-10 μg/ml. mAbGB3.1 and isotype control antibody (Srikrishna et al. (2001) J Immunol 166:624) were used at 40 μg/ml. Intracellular and cell surface staining were performed as described (Sinha et al. (2005) J Immunol 174:636). Stained samples were run on a BECKMAN COULTER® XL flow cytometer and analyzed using FCS-Express analysis software.

### Tumor Cells, Tumor Cell Inoculation Tumor Growth, Surgery, and Antibody Treatment

4T1 mouse mammary carcinoma cells were maintained as described (Pulaski et al. (1998) Cancer Res 58:1486). For tumor progression experiments, BALB/c mice were inoculated in the abdominal mammary gland with 7000 4T1 cells, and tumor progression, surgical removal of primary tumors, and quantification of metastatic disease were performed as described (Pulaski et al. (1998) Cancer Res 58:1486). mAbGB3.1 or isotype control antibody treatment (10 μg protein/gm body weight, i.v) was started 3 days after removal of primary tumor and continued once weekly. Seventy-two hrs after each treatment, mice were tail bled into 500 μl of a 0.008% heparin solution, and RBCs were removed by lysis (Sinha et al. (2007) J Immunol 179:977). Remaining white cells were identified by flow cytometry (Sinha et al. (2005) J Immunol 174:636). Ab treatment was terminated when mice were moribund and spleens, lymph nodes, and lungs were cryopreserved for immunohistochemistry.

### MDSC Purification, Phenotype, Morphology, and T Cell Suppression Assay

Spleens, lungs and femurs were obtained from 4T1 tumor-bearing mice on days 33 after inoculation of 7000 4T1 tumor cells. Femurs were flushed with excess PBS using a syringe fitted with a 27 g needle. Spleens and lungs were dissociated to single cell suspensions as described (Pulaski et al. (1998) Cancer Res 58:1486) Splenocytes, bone marrow and lung cells were depleted of RBCs, and MDSC were isolated by magnetic bead sorting of Gr1+cells using Miltenyi Biotec magenetic beads, as described (Sinha et al. (2005) J Immunol 174:636). For blood MDSC, tumor-bearing mice were bled into 500 μl of a 0.008% heparin solution, and RBC removed by lysis as described (Sinha et al. (2005) J Immunol 174:636). Remaining white blood cells that were >90% Gr1+CD11b+ were used in experiments. MDSC were phenotyped using fluorescent conjugated antibodies and stained with Wright-Giemsa stain using Diff-Quik (Dade Behring) (Sinha et al. (2005) J Immunol 174:636). T-cell suppression was assessed by co-culturing DOI 1.10 or clone 4 splenocytes with irradiated blood MDSC or MACS purified MDSC and specific peptide as described (Sinha et al. (2005) J Immunol 174:636).

### Real-Time RT-PCR Analysis

MDSC from tumor-bearing mice were isolated as described. To obtain sufficient Gr1⁺CD11b⁺ cells from tumor-free mice, mice were bled as described and Gr1⁺CD11b⁺ cells were purified using Gr1 antibody and Miltenyi magnetic beads (Sinha et al. (2005) J Immunol 174:636). The resulting cells were washed once with PBS and the pellets were frozen in dry ice, and sent to SuperArray, Inc. for RT-PCR analysis. Results are expressed as the number of cycles needed to detect product, or the fold change of tumor MDSC vs. naive MDSC as calculated by the delta delta ct method (Livak et al. (2001) Methods 25:402) using the following formulas:

Delta Ct=Average Ct (Experimental)−Average Ct (house keeping genes).

Fold change=2̂ (tumor MDSC delta ct−naïve MDSC delta ct).

The housekeeping gene panel (HG) included glucuronidase beta (Gusb), hypoxanthine guanine phosphoribosyl transferase 1 (Hprt1), heat shock protein 90 kDa alpha (cytosolic) class B member 1 (Hsp90ab1), glyceraldehyde-3-phosphate dehydrogenase (Gapdh), and actin, beta, cytoplasmic (Actb).

### Immunohistochemistry

Cryosections (6 μm) were air-dried, fixed in cold acetone for 2 min at room temperature, rehydrated in PBS, stained with commercially available anti-mouse antibodies to CD11b and Gr-1, followed by commercially available ALEXA FLUOR®594 or ALEXA FLUOR®488 conjugated secondary antibodies (Invitrogen), cover-slipped with commercially available mounting medium, and examined using an inverted TE300 NIKON® wide field fluorescence microscope and photographed with a CCD SPOT RT Camera (Diagnostic Instruments Inc.). Cells in three high power fields were counted for quantitation.

### Western Blots

Western blots were performed on 12% SDS-PAGE gels using equivalent amounts of cell lysate proteins of MACS purified MDSC. Ten percent skimmed milk was used for blocking. S100A8 and S100A9 were detected with goat polyclonal abs (R&D Systems) and alkaline phosphatase-conjugated anti-goat secondary antibody (Jackson Immunoresearch). Bands were visualized using commercially available BCIP®/NBT.

### S100A8 and S100A9 Quantification

Secreted S100A8 and S100A9 proteins were quantified by ELISA as described (Vandal et al. (2003) J Immunol 171:2602) with the following modifications: Coating antibody was goat-anti-mouse S100A9 mAb (R&D Systems). Culture supernatants of MDSC (Sinha et al. (2007) J Immunol 179:977) or 4T1 tumor or serum from tumor-bearing mice were diluted in HBSS containing 0.05% Tween-20. Polyclonal rabbit anti-mouse S100A8 Ab (Goebeler et al. (1993) J Leukoc Biol 53:11) was biotinylated using NHS-biotin as per the manufacturer's protocol (Pierce) and used at a dilution of 1:1000. Incubation with biotinylated anti-S100A8 antibody was for 2 hr at room temperature, followed by streptavidin alkaline phosphatase (Promega, 1:2000) for one hour at room temperature. Wells were developed with p-nitrophenyl phosphate substrate and read at 405 nm. Purified S100A8/A9 (Hunter et al. (1998) J Biol Chem 273:12427) served as standard.

### S100A8/A9 Protein Binding

Cell membranes were generated from 108 blood MDSC by homogenization in 10 mM Tris-HCl, pH 7.4, containing 200 mM sucrose and protease inhibitors (cocktail of EDTA-free serine and cysteine protease inhibitors, Roche). Nuclei and cell debris were removed by centrifugation at 900×g for 10 min at 4° C. Resulting supernatants were ultracentrifuged at 110,000×g for 75 min at 4° C., and pellets resuspended in 200 μl of 10 mM Tris-HCl, pH 7.4 with 150 mM NaCl and protease inhibitors. Endogenous S100A8/A9 proteins were stripped by incubation in 50 mM glycine, 100 mM NaCl pH 3.0 for 3 min at 4° C. followed by neutralization with cold HBSS. Membranes were incubated with increasing concentrations of Na125I-labeled purified S100A8/A9 (labeled using lodobeads (Pierce) as per the manufacturer's protocol to a specific activity of 2×105 cpm/pmole) for 1 hr at 4° C. in HBSS, washed twice with 1 ml of HBSS, recovered by ultracentrifugation, solubilized in 0.2 ml of 0.5 M NaOH, and cell-bound radioactivity was counted. Non-specific binding was determined by binding in the presence of 100-fold molar excess of cold ligand. Where required, binding was carried out in the presence of 10 fold molar excess of mAbGB3.1, anti-RAGE, anti-S100A8, or isotype-matched irrelevant mAb. Saturation binding kinetic analyses were performed using GRAPHPAD PRISM®. Values were normalized for number of cells.

### NF-κB Phosphorylation

Blood MDSC (108/ml) were cultured with S100A8/A9 (5 μg/ml) or TNFα (20 ng/ml), with or without mAb GB3.1 or control irrelevant mAb (10 μg/ml) in 250 μl of serum-free DMEM in 24 well plates. At the indicated times, MDSC were washed with cold PBS, lysed in 200 μl lysis buffer (Cell Signaling Technology) containing PMSF (1 mM) and protease inhibitors (1 tab/10 ml lysis buffer). Cell lysates were centrifuged at 4° C. and supernatants stored at −80° C. until analyzed by PathScan Inflammation Multi-Target Sandwich ELISA Kit or PathScan phospho-NF-kB p65 (Ser536) Sandwich ELISA Kit according to the manufacturer's protocol (Cell Signaling Technology). Percentage increase in pNF-κB=(100%)×[(MDSC with inducer)−(MDSC without inducer)/(MDSC without inducer)].

### Chemotaxis

In vitro migration of MDSC was evaluated in 24 well plates with transwell polycarbonate permeable supports (8.0 μm) (Costar Corning Inc, Corning N.Y.). One million MDSC (>90% Gr1+CD11b+ cells) were plated in 100 μl of serum free IMDM in the upper compartment and 500 μl of chemoattractant (tumor conditioned media ±10 μg of Abs to S100A8 or S100A9, or IgG control Ab) were added to the lower compartment. Plates were incubated at 37° C. with 5% CO2 for 3 hr, and the number of MDSC in the bottom compartment counted. For conditioned media (CM), supernatants were harvested from confluent cultures of 4T1 tumor cells cultured in medium IMDM containing 3% serum and 1% antibiotics. Conditioned media was filter sterilized and stored at −80° C. as single use aliquots. Percentage increase in migration of Gr1+CD11b+ cells=(100%)×[(MDSC migration with inducer±antibody)−(MDSC migration in media control)/( MDSC migration in media control)].

### EXAMPLE 15

### Treating Cancer in a Subject by Administrating an Agent that Inhibits Binding of an S100 Protein to RAGE

This example describes an approach that can be used to treat cancer in a subject. A subject is identified as having cancer (e.g., colon cancer). An appropriate dosage(s) of an antibody against RAGE (e.g., monoclonal antibody GB3.1) or a modified version thereof is administered to the subject. The antibody inhibits the binding of an S100 protein (e.g., S100A8 or S100A9) to RAGE. Optionally, an additional cancer therapy (e.g, chemotherapy, radiation, or a biological agent specific for the cancer) is provided to the patient. The subject is tested before and after administration of the antibody. After administration of the antibody, the subject will have an inhibition in proliferation of cancer cells or a reduction in the number of cancer cells or the size of the tumor compared to the subject before administration of the antibody.

### EXAMPLE 16

### Preventing Cancer in a Subject at High Risk for Cancer by Administrating an Agent that Inhibits Binding of an S100 Protein to RAGE

This example describes an approach that can be used to prevent cancer in a subject. A subject is identified as having a high risk for cancer (e.g., colon cancer). The subject is suffering from an inflammatory condition (e.g., colonic inflammation) that normally progresses to carcinogenesis or tumor formation. An appropriate dosage(s) of an antibody against RAGE (e.g., monoclonal antibody GB3.1) or a modified version thereof is provided to the subject. The antibody inhibits the binding of an S100 protein (e.g., S100A8 or S100A9) to RAGE. The subject is periodically tested after administration of the antibody. After administration of the antibody, the subject's inflammatory condition will not progress to carcinogenesis or tumor formation.

What is claimed is:

1. A method of treating cancer in a subject, comprising administrating to the subject an agent that inhibits binding of an S100 protein to a cell surface glycoprotein receptor.

2. The method of claim 1, wherein said cell surface glycoprotein receptor comprises Receptor for Advanced Glycation End-products (RAGE).

3. The method of claim 2, wherein said RAGE protein has been modified with a carboxylated glycan.

4. The method of claim 1, wherein said S100 protein comprises S100A8 or S100A9.

5. The method of claim 1, wherein said agent is an antibody.

6. The method of claim 5, wherein said antibody binds to Receptor for Advanced Glycation End-products (RAGE).

7. The method of claim 6, wherein said antibody is monoclonal antibody GB3.1.

8. The method of claim 1, wherein said cell surface glycoprotein receptor is expressed on a myeloid, monocytic, dendritic, endothelial, or tumor cell.

9. The method of claim 8, wherein said cell surface glycoprotein receptor is expressed on a myeloid-derived suppressor cell (MDSC).

10. The method of claim 1, further comprising providing a cancer therapy selected from the group consisting of a chemotherapeutic agent, radiation, and a biological agent specific for a cancer cell.

11. A method of preventing cancer in a subject comprising:
identifying a subject with a high risk for cancer; and
providing said subject an agent that binds to a cell surface glycoprotein receptor or an S100 protein,
wherein the binding of said agent to said cell surface glycoprotein receptor or said S100 protein inhibits the binding of said S100 protein to said cell surface glycoprotein receptor.

12. The method of claim 11, wherein said cell surface glycoprotein receptor comprises Receptor for Advanced Glycation End-products (RAGE).

13. The method of claim 12, wherein said RAGE protein has been modified with a carboxylated glycan.

14. The method of claim 11, wherein said S100 protein comprises S100A8 or S100A9.

15. The method of claim 11, wherein said agent is an antibody.

16. The method of claim 15, wherein said antibody is monoclonal antibody GB3.1.

17. The method of claim 11, wherein said cell surface glycoprotein receptor is expressed on a myeloid, monocytic, dendritic, endothelial, or tumor cell.

18. The method of claim 17, wherein said cell surface glycoprotein receptor is expressed on a myeloid-derived suppressor cell (MDSC).

19. The method of claim 11, wherein said subject with a high risk for cancer is suffering from an inflammatory condition.

20. The method of claim 19, wherein said inflammatory condition is colonic inflammation.

21. The method of claim 11, further comprising providing the subject with a cancer therapy selected from the group consisting of a chemotherapeutic agent, radiation, and a biological agent specific for a cancer cell.
